SEQUENCE LISTING (13 sequences)

SEQ ID NO: 1 - DNA, 1929 bp, synthetic construct
  source 1..1929: mol_type = other DNA; note = codon optimized for feline
atggagtcaccaacacaccctaaaccttctaaagacaaaaccctctcgtggaatctcgccttccttgtgggcatcctgttcacaatcgacatcggcatggccaacccttcgccgcatcagatctacaatgtgacatgggtcattactaatgtgcagacaaacacccaggcaaatgctacttctatgcttggtactctgactgatgcttatccaaccctgcacgtcgacctttgcgatctcgtcggtgacacatgggagcccatcgtgctgaatccaactaatgtcaaacatggtgccaggtattcttctagcaaatacgggtgtaagaccactgatcggaagaaacagcaacaaacctacccattctacgtgtgcccgggtcacgcaccgtccctgggtccgaagggaacacattgtgggggagcccaagacggtttttgcgctgcttggggttgtgaaacaaccggagaagcctggtggaagcctacctcatcttgggactacattactgtgaaaagaggctctagccaggataacagctgcgaaggaaagtgtaatcccctggtgcttcaattcacccagaaaggccggcaggcatcatgggatggaccgaaaatgtggggacttagactctatcgcaccggatacgaccccatcgctctgtttactgtgtcacgccaagtctccaccattactccgccacaggccatggggccgaatctggtcctccccgatcagaagccaccctcacggcaaagtcaaaccggctcaaaagtggccacccaacggccccagacaaatgagtccgcacctaggtcagtggcacctacaacaatgggtccaaagcggatcggaaccggagacaggctcattaacctcgtgcaagggacttatctggcccttaacgctactgaccccaacaagaccaaggattgctggctctgccttgtgagcagacctccttactatgaggggatcgccattctcggaaactactcaaatcagaccaacccccctccgtcgtgtctgagcaccccccagcacaagcttactatttcagaagtcagtggacagggaatgtgcatcggaaccgtgccaaagactcatcaagccctttgcaacaaaactcaacaagggcacactggagctcattatctcgccgcacctaacgggacctactgggcttgcaatactggattgaccccgtgtatctctatggccgtgctgaattggacttccgacttctgcgtgcttattgagctttggcctagagtgacataccatcagcctgagtacgtctatacccatttcgccaaggcagtcagattccggcgggagcctatctccctgactgtggccttgatgctcggtggactgacagtgggaggaattgcagctggagtcggaactggaaccaaggccctgctcgaaactgctcagttccggcagctgcagatggccatgcacactgacatccaggctctggaggaatcaatttcagcccttgagaaaagcttgacctcgctgtctgaagtggtcctccaaaacaggcgcggtttggacatcctgttccttcaagagggtggtctgtgcgccgctctcaaggaggaatgctgtttctacgctgaccataccgggctggtgcgcgataacatggcaaagctgcgggaacgcttgaaacagaggcagcaactgttcgactctcagcagggatggttcgagggctggtttaacaagagcccatggtttaccactctgatctcttcaatcatgggtccactgctcatcctgcttctgattcttctcttcggaccgtgtattctcaacaggctggtgcagtttgtcaaggacagaatctcggtggtccaggccctgattcttactcagcagtatcagcagattaagcagtacgaccccgatcggccttga

SEQ ID NO: 2 - protein, 642 aa, Feline leukemia virus
MESPTHPKPSKDKTLSWNLAFLVGILFTIDIGMANPSPHQIYNVTWVITNVQTNTQANATSMLGTLTDAYPTLHVDLCDLVGDTWEPIVLNPTNVKHGARYSSSKYGCKTTDRKKQQQTYPFYVCPGHAPSLGPKGTHCGGAQDGFCAAWGCETTGEAWWKPTSSWDYITVKRGSSQDNSCEGKCNPLVLQFTQKGRQASWDGPKMWGLRLYRTGYDPIALFTVSRQVSTITPPQAMGPNLVLPDQKPPSRQSQTGSKVATQRPQTNESAPRSVAPTTMGPKRIGTGDRLINLVQGTYLALNATDPNKTKDCWLCLVSRPPYYEGIAILGNYSNQTNPPPSCLSTPQHKLTISEVSGQGMCIGTVPKTHQALCNKTQQGHTGAHYLAAPNGTYWACNTGLTPCISMAVLNWTSDFCVLIELWPRVTYHQPEYVYTHFAKAVRFRREPISLTVALMLGGLTVGGIAAGVGTGTKALLETAQFRQLQMAMHTDIQALEESISALEKSLTSLSEVVLQNRRGLDILFLQEGGLCAALKEECCFYADHTGLVRDNMAKLRERLKQRQQLFDSQQGWFEGWFNKSPWFTTLISSIMGPLLILLLILLFGPCILNRLVQFVKDRISVVQALILTQQYQQIKQYDPDRP

SEQ ID NO: 3 - DNA, 1233 bp, synthetic construct
  source 1..1233: mol_type = other DNA; note = condon optimized for feline
aatcctagtccacaccaaatatataatgtaacttgggtaataaccaatgtacaaactaacacccaagctaacgccacctctatgttaggaaccttaaccgatgcctaccctaccctacatgttgacttatgtgacctagtgggagacacctgggaacctatagtcctaaacccaaccaatgtaaaacacggggcacgttactcctcctcaaaatatggatgtaaaactacagatagaaaaaaacagcaacagacataccccttttacgtctgccccggacatgccccctcgttggggccaaagggaacacattgtggaggggcacaagatgggttttgtgccgcatggggatgtgagaccaccggagaagcttggtggaagcccacctcctcatgggactatatcacagtaaaaagagggagtagtcaggacaatagctgtgagggaaaatgcaaccccctggttttgcagttcacccagaagggaagacaagcctcttgggacggacctaagatgtggggattgcgactataccgtacaggatatgaccctatcgctttattcacggtgtcccggcaggtatcaaccattacgccgcctcaggcaatgggaccaaacctagtcttacctgatcaaaaacccccatcccgacaatctcaaacagggtccaaagtggcgacccagaggccccaaacgaatgaaagcgccccaaggtctgttgcccccaccaccatgggtcccaaacggattgggaccggagataggttaataaatttagtacaagggacatacctagccttaaatgccaccgaccccaacaaaactaaagactgttggctctgcctggtttctcgaccaccctattacgaagggattgcaatcttaggtaactacagcaaccaaacaaacccccccccatcctgcctatctactccgcaacacaaactaactatatctgaagtatcagggcaaggaatgtgcatagggactgttcctaaaacccaccaggctttgtgcaataagacacaacagggacatacaggggcgcactatctagccgcccccaacggcacctattgggcctgtaacactggactcaccccatgcatttccatggcggtgctcaattggacctctgatttttgtgtcttaatcgaattatggcccagagtgacttaccatcaacccgaatatgtgtacacacattttgccaaagctgtcaggttccgaaga

SEQ ID NO: 4 - protein, 411 aa, Feline leukemia virus
NPSPHQIYNVTWVITNVQTNTQANATSMLGTLTDAYPTLHVDLCDLVGDTWEPIVLNPTNVKHGARYSSSKYGCKTTDRKKQQQTYPFYVCPGHAPSLGPKGTHCGGAQDGFCAAWGCETTGEAWWKPTSSWDYITVKRGSSQDNSCEGKCNPLVLQFTQKGRQASWDGPKMWGLRLYRTGYDPIALFTVSRQVSTITPPQAMGPNLVLPDQKPPSRQSQTGSKVATQRPQTNESAPRSVAPTTMGPKRIGTGDRLINLVQGTYLALNATDPNKTKDCWLCLVSRPPYYEGIAILGNYSNQTNPPPSCLSTPQHKLTISEVSGQGMCIGTVPKTHQALCNKTQQGHTGAHYLAAPNGTYWACNTGLTPCISMAVLNWTSDFCVLIELWPRVTYHQPEYVYTHFAKAVRFRR

SEQ ID NO: 5 - DNA, 1638 bp, synthetic construct
  source 1..1638: mol_type = other DNA; note = codon optimized for feline
atggctgacgacggatctgtgaccaccccagaacaaggaacaatggtcggaggagtgattgccgaacccagcgctcagatgtcaactgcggcggacatggcctccggaaagtcggtggactccgagtgggaagccttcttctcgttccacacgtccgtgaactggagcacctccgaaacccaaggaaagatcctcttcaagcagtccctgggtcccctgctgaacccgtacctggagcacatcagcaagctgtacgtcgcttggagcgggtcgatcgaagtgcgattttccatctcgggaagcggcgtgttcggtggtaaactggccgccatcgtcgtgccgcctggtgtcgaccctgtccagtcaacctccatgctgcagtacccgcacgtcctgttcgacgcaagacaagtggagccagtgatcttctccatcccggacctccgcaacagcctgtatcacttgatgtccgataccgataccacttccctcgtgatcatggtgtacaacgatctgatcaacccgtacgccaatgactccaacagctcgggttgcatcgtgaccgtcgaaacgaagcctggcatcgatttcaagtttcatctgctgaaaccgcccggatccatgcttactcacgggtccatcccttccgatctgatccccaagagctcctccctgtggattgggaaccgccactggaccgatattaccgatttcgtgattcggcctttcgtgttccaagccaaccggcacttcgacttcaaccaggagactgccggctggtcaactccacggttccgcccattggccgtgactgtgtcgcagtcaaagggagccaagctcgggaacggcatcgccaccgactacattgtgcctggaatccccgacggatggcctgatactaccatccccaccaagctgacccctaccggagattacgccatcacctcctccgacggcaatgatattgaaaccaagctggaatacgagaacgcggacgtgattaagaacaacaccaacttccgctccatgtatatctgcggaagcctccagagggcttggggcgacaagaagatcagcaacaccgggttcatcactaccggagtgatttctgacaactccatcagcccttcgaacacaattgaccagtccaagatcgtggtgtaccaggacaaccatgtcaattcggaggtccagactagcgacatcactcttgccatcctgggctacaccggaattggagaagaggccataggcgccaaccgggactccgtcgtgagaatttccgtgcttccggaaactggagcaaggggcggaaatcaccccatcttctacaaaaattccatgaagctgggctacgtgatctcctccattgacgtgttcaactcccaaatcctccacacctcgcgccagctgtcactgaacaactacttgttgccccctgactccttcgcggtgtaccggattattgacagcaacggatcatggttcgacattgggattgacagcgatgggttttcattcgtgggcgtgtcgtcatttccaaagctggagtttccgctgtccgcctcatacatgggcatccagctcgcaaagatccggctggcgtccaacatccggtcatccatgactaagctgtga

SEQ ID NO: 6 - protein, 545 aa, Feline calicivirus
MADDGSVTTPEQGTMVGGVIAEPSAQMSTAADMASGKSVDSEWEAFFSFHTSVNWSTSETQGKILFKQSLGPLLNPYLEHISKLYVAWSGSIEVRFSISGSGVFGGKLAAIVVPPGVDPVQSTSMLQYPHVLFDARQVEPVIFSIPDLRNSLYHLMSDTDTTSLVIMVYNDLINPYANDSNSSGCIVTVETKPGIDFKFHLLKPPGSMLTHGSIPSDLIPKSSSLWIGNRHWTDITDFVIRPFVFQANRHFDFNQETAGWSTPRFRPLAVTVSQSKGAKLGNGIATDYIVPGIPDGWPDTTIPTKLTPTGDYAITSSDGNDIETKLEYENADVIKNNTNFRSMYICGSLQRAWGDKKISNTGFITTGVISDNSISPSNTIDQSKIVVYQDNHVNSEVQTSDITLAILGYTGIGEEAIGANRDSVVRISVLPETGARGGNHPIFYKNSMKLGYVISSIDVFNSQILHTSRQLSLNNYLLPPDSFAVYRIIDSNGSWFDIGIDSDGFSFVGVSSFPKLEFPLSASYMGIQLAKIRLASNIRSSMTKL

SEQ ID NO: 7 - DNA, 1629 bp, synthetic construct
  source 1..1629: mol_type = other DNA; note = codon optimized for feline
atgactgccccggaacaaggaacgatggtcggaggagtgattgcagaaccgtcagcacagatgtccaccgctgccgacatggccactggaaagagcgtggactccgaatgggaagccttcttctccttccacacttcggtcaactggtcgactagcgaaacccaggggaagattttgttcaagcaatccctcggccctctgctgaacccctacctggagcatctggccaagctgtacgtggcatggtcgggcagcatcgaagtgcgctttagcatttccggctccggagtgttcgggggaaagcttgctgccattgtcgtgccgccaggagtggacccggtgcagtccacttctatgctccaatacccgcatgtcctgttcgacgccagacaggtggagcctgtgatcttttgcctgccggatctcaggtccaccctgtatcacctcatgtccgacaccgacaccacctcgctcgtgatcatggtgtacaacgacctgatcaacccctacgctaacgacgccaacagctcaggttgcattgtgactgtcgaaaccaagccaggccctgacttcaagtttcatttgctgaagccgcccggttccatgctgacccacggctcgatcccatccgacctgatccccaagacgagctccctgtggatcggaaaccgctactggtccgatattaccgacttcgtgatcagaccattcgtgttccaagccaaccgccatttcgacttcaaccaggaaaccgcaggatggtcgacccctcgattccgcccgatttcagtgaccatcaccgaacagaacggcgcgaagctgggaattggcgtggcgaccgactacatcgtgccgggaatcccggatggatggcctgatacgaccattcccggggagctgatccctgccggggactacgccatcaccaacggtactggaaacgacatcaccactgccaccggttacgacaccgccgacatcataaagaacaacaccaacttcagaggaatgtacatttgcggctccctgcaacgcgcttggggtgacaaaaagatctcgaacactgccttcatcacaacagcgactctggacggcgataacaacaacaagatcaatccttgtaataccatcgaccagtccaaaatcgtggtgttccaggataaccacgtgggaaagaaggcgcagacctccgacgacactctggcgctgcttggctacaccgggatcggcgagcaggccattggaagcgatcgggatcgggtcgtgcggatctccaccctccccgagactggagcaaggggaggcaaccaccccatcttttacaaaaacagcattaagctcggatacgtcatccgctccatcgatgtgttcaactctcaaatcctgcacacttcgcggcagctgtccctgaaccactacctcttgccgcccgactccttcgccgtctaccggatcattgattcgaacgggagctggttcgacatcggcattgatagcgatggcttctcgtttgtgggcgtgtcgggcttcgggaagctggagttcccactgagcgcctcatacatgggtatccagctggccaagatcaggctggcctccaacatccgctcacctatgactaagctgtga

SEQ ID NO: 8 - protein, 542 aa, Feline calicivirus
MTAPEQGTMVGGVIAEPSAQMSTAADMATGKSVDSEWEAFFSFHTSVNWSTSETQGKILFKQSLGPLLNPYLEHLAKLYVAWSGSIEVRFSISGSGVFGGKLAAIVVPPGVDPVQSTSMLQYPHVLFDARQVEPVIFCLPDLRSTLYHLMSDTDTTSLVIMVYNDLINPYANDANSSGCIVTVETKPGPDFKFHLLKPPGSMLTHGSIPSDLIPKTSSLWIGNRYWSDITDFVIRPFVFQANRHFDFNQETAGWSTPRFRPISVTITEQNGAKLGIGVATDYIVPGIPDGWPDTTIPGELIPAGDYAITNGTGNDITTATGYDTADIIKNNTNFRGMYICGSLQRAWGDKKISNTAFITTATLDGDNNNKINPCNTIDQSKIVVFQDNHVGKKAQTSDDTLALLGYTGIGEQAIGSDRDRVVRISTLPETGARGGNHPIFYKNSIKLGYVIRSIDVFNSQILHTSRQLSLNHYLLPPDSFAVYRIIDSNGSWFDIGIDSDGFSFVGVSGFGKLEFPLSASYMGIQLAKIRLASNIRSPMTKL

SEQ ID NO: 9 - DNA, 13 bp, synthetic construct
  source 1..13: mol_type = other DNA; note = 5' flanking sequence
ggcgcgccgcacc

SEQ ID NO: 10 - RNA, 1929 nt, synthetic construct
  source 1..1929: mol_type = other RNA; note = codon optimized for feline
atggagtcaccaacacaccctaaaccttctaaagacaaaaccctctcgtggaatctcgccttccttgtgggcatcctgttcacaatcgacatcggcatggccaacccttcgccgcatcagatctacaatgtgacatgggtcattactaatgtgcagacaaacacccaggcaaatgctacttctatgcttggtactctgactgatgcttatccaaccctgcacgtcgacctttgcgatctcgtcggtgacacatgggagcccatcgtgctgaatccaactaatgtcaaacatggtgccaggtattcttctagcaaatacgggtgtaagaccactgatcggaagaaacagcaacaaacctacccattctacgtgtgcccgggtcacgcaccgtccctgggtccgaagggaacacattgtgggggagcccaagacggtttttgcgctgcttggggttgtgaaacaaccggagaagcctggtggaagcctacctcatcttgggactacattactgtgaaaagaggctctagccaggataacagctgcgaaggaaagtgtaatcccctggtgcttcaattcacccagaaaggccggcaggcatcatgggatggaccgaaaatgtggggacttagactctatcgcaccggatacgaccccatcgctctgtttactgtgtcacgccaagtctccaccattactccgccacaggccatggggccgaatctggtcctccccgatcagaagccaccctcacggcaaagtcaaaccggctcaaaagtggccacccaacggccccagacaaatgagtccgcacctaggtcagtggcacctacaacaatgggtccaaagcggatcggaaccggagacaggctcattaacctcgtgcaagggacttatctggcccttaacgctactgaccccaacaagaccaaggattgctggctctgccttgtgagcagacctccttactatgaggggatcgccattctcggaaactactcaaatcagaccaacccccctccgtcgtgtctgagcaccccccagcacaagcttactatttcagaagtcagtggacagggaatgtgcatcggaaccgtgccaaagactcatcaagccctttgcaacaaaactcaacaagggcacactggagctcattatctcgccgcacctaacgggacctactgggcttgcaatactggattgaccccgtgtatctctatggccgtgctgaattggacttccgacttctgcgtgcttattgagctttggcctagagtgacataccatcagcctgagtacgtctatacccatttcgccaaggcagtcagattccggcgggagcctatctccctgactgtggccttgatgctcggtggactgacagtgggaggaattgcagctggagtcggaactggaaccaaggccctgctcgaaactgctcagttccggcagctgcagatggccatgcacactgacatccaggctctggaggaatcaatttcagcccttgagaaaagcttgacctcgctgtctgaagtggtcctccaaaacaggcgcggtttggacatcctgttccttcaagagggtggtctgtgcgccgctctcaaggaggaatgctgtttctacgctgaccataccgggctggtgcgcgataacatggcaaagctgcgggaacgcttgaaacagaggcagcaactgttcgactctcagcagggatggttcgagggctggtttaacaagagcccatggtttaccactctgatctcttcaatcatgggtccactgctcatcctgcttctgattcttctcttcggaccgtgtattctcaacaggctggtgcagtttgtcaaggacagaatctcggtggtccaggccctgattcttactcagcagtatcagcagattaagcagtacgaccccgatcggccttga

SEQ ID NO: 11 - RNA, 1233 nt, synthetic construct
  source 1..1233: mol_type = other RNA; note = codon optimized for feline
aatcctagtccacaccaaatatataatgtaacttgggtaataaccaatgtacaaactaacacccaagctaacgccacctctatgttaggaaccttaaccgatgcctaccctaccctacatgttgacttatgtgacctagtgggagacacctgggaacctatagtcctaaacccaaccaatgtaaaacacggggcacgttactcctcctcaaaatatggatgtaaaactacagatagaaaaaaacagcaacagacataccccttttacgtctgccccggacatgccccctcgttggggccaaagggaacacattgtggaggggcacaagatgggttttgtgccgcatggggatgtgagaccaccggagaagcttggtggaagcccacctcctcatgggactatatcacagtaaaaagagggagtagtcaggacaatagctgtgagggaaaatgcaaccccctggttttgcagttcacccagaagggaagacaagcctcttgggacggacctaagatgtggggattgcgactataccgtacaggatatgaccctatcgctttattcacggtgtcccggcaggtatcaaccattacgccgcctcaggcaatgggaccaaacctagtcttacctgatcaaaaacccccatcccgacaatctcaaacagggtccaaagtggcgacccagaggccccaaacgaatgaaagcgccccaaggtctgttgcccccaccaccatgggtcccaaacggattgggaccggagataggttaataaatttagtacaagggacatacctagccttaaatgccaccgaccccaacaaaactaaagactgttggctctgcctggtttctcgaccaccctattacgaagggattgcaatcttaggtaactacagcaaccaaacaaacccccccccatcctgcctatctactccgcaacacaaactaactatatctgaagtatcagggcaaggaatgtgcatagggactgttcctaaaacccaccaggctttgtgcaataagacacaacagggacatacaggggcgcactatctagccgcccccaacggcacctattgggcctgtaacactggactcaccccatgcatttccatggcggtgctcaattggacctctgatttttgtgtcttaatcgaattatggcccagagtgacttaccatcaacccgaatatgtgtacacacattttgccaaagctgtcaggttccgaaga

SEQ ID NO: 12 - RNA, 1638 nt, synthetic construct
  source 1..1638: mol_type = other RNA; note = codon optimized for feline
atggctgacgacggatctgtgaccaccccagaacaaggaacaatggtcggaggagtgattgccgaacccagcgctcagatgtcaactgcggcggacatggcctccggaaagtcggtggactccgagtgggaagccttcttctcgttccacacgtccgtgaactggagcacctccgaaacccaaggaaagatcctcttcaagcagtccctgggtcccctgctgaacccgtacctggagcacatcagcaagctgtacgtcgcttggagcgggtcgatcgaagtgcgattttccatctcgggaagcggcgtgttcggtggtaaactggccgccatcgtcgtgccgcctggtgtcgaccctgtccagtcaacctccatgctgcagtacccgcacgtcctgttcgacgcaagacaagtggagccagtgatcttctccatcccggacctccgcaacagcctgtatcacttgatgtccgataccgataccacttccctcgtgatcatggtgtacaacgatctgatcaacccgtacgccaatgactccaacagctcgggttgcatcgtgaccgtcgaaacgaagcctggcatcgatttcaagtttcatctgctgaaaccgcccggatccatgcttactcacgggtccatcccttccgatctgatccccaagagctcctccctgtggattgggaaccgccactggaccgatattaccgatttcgtgattcggcctttcgtgttccaagccaaccggcacttcgacttcaaccaggagactgccggctggtcaactccacggttccgcccattggccgtgactgtgtcgcagtcaaagggagccaagctcgggaacggcatcgccaccgactacattgtgcctggaatccccgacggatggcctgatactaccatccccaccaagctgacccctaccggagattacgccatcacctcctccgacggcaatgatattgaaaccaagctggaatacgagaacgcggacgtgattaagaacaacaccaacttccgctccatgtatatctgcggaagcctccagagggcttggggcgacaagaagatcagcaacaccgggttcatcactaccggagtgatttctgacaactccatcagcccttcgaacacaattgaccagtccaagatcgtggtgtaccaggacaaccatgtcaattcggaggtccagactagcgacatcactcttgccatcctgggctacaccggaattggagaagaggccataggcgccaaccgggactccgtcgtgagaatttccgtgcttccggaaactggagcaaggggcggaaatcaccccatcttctacaaaaattccatgaagctgggctacgtgatctcctccattgacgtgttcaactcccaaatcctccacacctcgcgccagctgtcactgaacaactacttgttgccccctgactccttcgcggtgtaccggattattgacagcaacggatcatggttcgacattgggattgacagcgatgggttttcattcgtgggcgtgtcgtcatttccaaagctggagtttccgctgtccgcctcatacatgggcatccagctcgcaaagatccggctggcgtccaacatccggtcatccatgactaagctgtga

SEQ ID NO: 13 - RNA, 1629 nt, synthetic construct
  source 1..1629: mol_type = other RNA; note = codon optimized for feline
atgactgccccggaacaaggaacgatggtcggaggagtgattgcagaaccgtcagcacagatgtccaccgctgccgacatggccactggaaagagcgtggactccgaatgggaagccttcttctccttccacacttcggtcaactggtcgactagcgaaacccaggggaagattttgttcaagcaatccctcggccctctgctgaacccctacctggagcatctggccaagctgtacgtggcatggtcgggcagcatcgaagtgcgctttagcatttccggctccggagtgttcgggggaaagcttgctgccattgtcgtgccgccaggagtggacccggtgcagtccacttctatgctccaatacccgcatgtcctgttcgacgccagacaggtggagcctgtgatcttttgcctgccggatctcaggtccaccctgtatcacctcatgtccgacaccgacaccacctcgctcgtgatcatggtgtacaacgacctgatcaacccctacgctaacgacgccaacagctcaggttgcattgtgactgtcgaaaccaagccaggccctgacttcaagtttcatttgctgaagccgcccggttccatgctgacccacggctcgatcccatccgacctgatccccaagacgagctccctgtggatcggaaaccgctactggtccgatattaccgacttcgtgatcagaccattcgtgttccaagccaaccgccatttcgacttcaaccaggaaaccgcaggatggtcgacccctcgattccgcccgatttcagtgaccatcaccgaacagaacggcgcgaagctgggaattggcgtggcgaccgactacatcgtgccgggaatcccggatggatggcctgatacgaccattcccggggagctgatccctgccggggactacgccatcaccaacggtactggaaacgacatcaccactgccaccggttacgacaccgccgacatcataaagaacaacaccaacttcagaggaatgtacatttgcggctccctgcaacgcgcttggggtgacaaaaagatctcgaacactgccttcatcacaacagcgactctggacggcgataacaacaacaagatcaatccttgtaataccatcgaccagtccaaaatcgtggtgttccaggataaccacgtgggaaagaaggcgcagacctccgacgacactctggcgctgcttggctacaccgggatcggcgagcaggccattggaagcgatcgggatcgggtcgtgcggatctccaccctccccgagactggagcaaggggaggcaaccaccccatcttttacaaaaacagcattaagctcggatacgtcatccgctccatcgatgtgttcaactctcaaatcctgcacacttcgcggcagctgtccctgaaccactacctcttgccgcccgactccttcgccgtctaccggatcattgattcgaacgggagctggttcgacatcggcattgatagcgatggcttctcgtttgtgggcgtgtcgggcttcgggaagctggagttcccactgagcgcctcatacatgggtatccagctggccaagatcaggctggcctccaacatccgctcacctatgactaagctgtga

(19) United States
(12) Patent Application Publication
(10) Pub. No.: US 2024/0398934 A1
(43) Pub. Date: Dec. 5, 2024

(54) FELINE LEUKEMIA VIRUS VACCINE

(71) Applicant: INTERVET INC., Rahway, NJ (US)

(72) Inventor: Ian TARPEY, St. Ives (GB)

(73) Assignee: INTERVET INC., Rahway, NJ (US)

(21) Appl. No.: 18/794,860

(22) Filed: Aug. 5, 2024

Related U.S. Application Data
(63) Continuation of application No. 17/698,205, filed on Mar. 18, 2022, now Pat. No. 12,076,392.
Application No. 17/698,205 is a continuation of application No. 16/759,927, filed on Apr. 28, 2020, now Pat. No. 11,311,616.
(60) Provisional application No. 62/599,401, filed on Dec. 15, 2017.
(60) Provisional application No. 62/596,508, filed on Dec. 8, 2017.
(60) Provisional application No. 62/582,050, filed on Nov. 6, 2017.

(51) Int. Cl. A61K 39/21; A61K 39/00; A61P 31/14; C12N 7/00; C12N 15/86

(52) U.S. Cl. CPC A61K 39/21; A61P 31/14; C12N 7/00; C12N 15/86; A61K 2039/5256; A61K 2039/5258; A61K 2039/545; A61K 2039/552; A61K 2039/70; C12N 2740/13034; C12N 2740/13071; C12N 2770/36143

(57) ABSTRACT

The present invention provides a vaccine for feline leukemia virus and methods of making and using the vaccine alone, or in combinations with other protective agents.

### CROSS-REFERENCE TO RELATED APPLICATIONS

This application claims priority under 35 U.S.C. § 119 (e) of provisional applications U.S. Ser. No. 62/582,050 filed Nov. 6, 2017, U.S. Ser. No. 62/596,508 filed Dec. 8, 2017, and U.S. Ser. No. 62/599,401 filed Dec. 15, 2017, the content of all of which are hereby incorporated by reference in their entireties.

### FIELD OF THE INVENTION

The present invention relates to new vaccines for feline leukemia virus. Methods of making and using the vaccine alone or in combinations with other protective agents are also provided.

### BACKGROUND

Feline leukemia virus (FeLV) is a retrovirus that infects domestic cats, resulting in significant morbidity and mortality worldwide. Though predominantly transmitted through saliva, FeLV also has been reported to spread through contact with body fluids [Pacitti et al., Vet Rec 118:381-384 (1986) doi: 10.1136/vr.118.14.381; Levy et al., J Feline Med Surg 10:300-316 (2008) doi: 10.1016/j.jfms.2008.03.002]. The clinical signs in cats observed during FeLV infections include: cytoproliferative disorders (lymphoid or myeloid tumors), cytosuppressive disorders (infectious diseases associated with immunosuppression, anemia, myelosuppression), inflammatory disorders, neurological disorders, abortions, and enteritis [Hoover et al., J Am Vet Med Assoc 199:1287-1297 (1991); Levy and Crawford, Textbook of Veterinary Internal Medicine, 6th ed (Ettinger S J, Feldman E C., eds.) WB Saunders, Philadelphia, PA. (2005)]. The prevalence of antigenemia may vary from 1-5% in healthy cats to 15-30% in afflicted cats [Hosie et al. Veterinary Records, 128:293-297 (1989); Braley, Feline Practice 22:25-29 (1994); Malik et al., Australian Veterinary Journal 75:323-327 (1997); Arjona et al., Journal of Clinical Microbiology 38:3448-3449 (2000)]. FeLV frequently establishes a lasting infection with a concomitant persistent viremia, often leading to the death of the host cat.

The RNA genome of FeLV encodes only three genes: (i) an ENV gene, which encodes the envelope glycoprotein, (ii) a GAG gene, which encodes the major structural components of the virus, and (iii) a POL gene, which encodes the RNA polymerase [Thomsen et al., Journal of General Virology 73:1819-1824 (1992)]. The FeLV envelope (ENV) gene encodes a gp85 precursor protein which is proteolytically processed by one or more cellular enzymes to yield the major envelope glycoprotein gp70 and the associated transmembrane protein p15E [DeNoronha, et al., Virology 85:617-621 (1978); Nunberg et al., PNAS 81:3675-3679 (1983)]. The transmembrane protein p15E contains a sequence conserved among gammaretroviruses with immunosuppressive properties [Mathes et al., Nature 274:687-689 (1978)]. Recently, The European Medicines Agency's Committee for Medicinal Products for Veterinary Use (CVMP) has adopted a positive opinion for a vaccine comprising a recombinant p45 FeLV-envelope antigen derived from the gp70 surface glycoprotein of the FeLV subgroup A that is expressed in Escherichia coli as active substance. The FeLV envelope glycoprotein is the target of FeLV-specific cytotoxic T cell responses, as well as neutralizing antibodies and accordingly, one of the major immunogens of FELV [Flynn et al., J. Virol. 76 (5): 2306-2315 (2002)].

A variety of factors, such as the immune status of the host, the age of the host, the infecting FeLV strain, the viral load, as well as the route of the exposure of the FeLV can all affect the ultimate outcome of that exposure. At one time veterinarians and researchers classified FeLV infections in relation to the relative persistence of the concomitant antigenemia, which in the more fortunate cases were a transient antigenemia and/or the elimination of infection. Assays for antigenemia include p27 enzyme-linked immunosorbent assay (ELISA), virus isolation, and immunofluorescence assays [Hoover et al., J Am Vet Med Assoc 199:1287-1297 (1991); Rojko and Kociba, J Am Vet Med Assoc 199:1305-1310 (1991)]. Such tests remain helpful in clinical applications, as well as for determining whether the clinical disease is a result of an actively circulating virus.

Four vaccines for FeLV are currently available in the United States, including two whole-virus adjuvanted killed vaccines; a dual-adjuvanted, multiple-antigen vaccine; and a nonadjuvanted, canarypox virus-vectored vaccine. Notably, different vaccines have been shown to have various degrees of efficacy [Sparkes, J Small Anim Pract 38:187-194 (1997) doi: 10.1111/j.1748-5827.1997.tb03339.x.]. Earlier studies demonstrated the efficacy of whole-virus adjuvanted killed vaccines after challenge, including testing vaccinated and unvaccinated cats for viral RNA, proviral DNA, FeLV antibodies, and the p27 antigen [Hines et al., J Am Vet Med Assoc 199:1428-1430 (1991); Pedersen, J Vet Intern Med 7:34-39 (1993) doi: 10.1111/j.1939-1676.1993.tb03166.x.]; Torres et al., Vet Immunol Immunopathol 134:122-131 (2010) doi: 10.1016/j.vetimm.2009.10.017]. There are limited data evaluating the efficacy of the nonadjuvanted recombinant FELV vaccine available for use [Stuke et al., Vaccine 32:2599-2603 (2014) doi: 10.1016/j.vaccine.2014.03.016].

More recently, the efficacy of two commercially available feline leukemia vaccines, one an inactivated whole-virus vaccine and the other a live canarypox virus-vectored vaccine have been compared following a challenge with virulent feline leukemia virus [Patel et al., Clinical and Vaccine Immunology 22 (7): 798-805 (2015)]. In this study the whole-virus adjuvanted killed vaccine was again found to provide superior protection against FeLV infection. However, the use of whole-virus killed adjuvanted FeLV vaccines has been implicated as one factor that leads to the development of feline injection-site sarcomas [Kass et al., J. AM Vet Med Assoc 203 (3): 396-405 (1993)]. Although subsequent studies have been unable to establish a direct link between killed adjuvanted vaccines and feline injection-site sarcomas, a perception remains that nonadjuvanted vaccines are safer. Indeed, the American Association of Feline Practitioners Feline Vaccination Guidelines suggest the use of nonadjuvanted FeLV vaccines to lower the risk of feline injection-site sarcomas and to reduce local inflammation [AAFP Feline Advisory Panel, 15:785-808 (2013)].

A number of vector strategies have been employed through the years for vaccines in an effort to protect against certain pathogens. One such vector strategy includes the use of alphavirus-derived replicon RNA particles (RP) [Vander Veen, et al. Anim Health Res Rev. 13 (1): 1-9. (2012) doi: 10.1017/S1466252312000011; Kamrud et al., J Gen Virol. 91 (Pt 7): 1723-1727 (2010)] which have been developed from several different alphaviruses, including Venezuelan equine encephalitis virus (VEE) [Pushko et al., Virology 239:389-401 (1997)], Sindbis (SIN) [Bredenbeck et al., Journal of Virology 67:6439-6446 (1993)], and Semliki Forest virus (SFV) [Liljestrom and Garoff, Biotechnology (NY) 9:1356-1361 (1991)]. RP vaccines deliver propagation-defective alphavirus RNA replicons into host cells and result in the expression of the desired antigenic transgene(s) in vivo [Pushko et al., Virology 239 (2): 389-401 (1997)]. RPs have an attractive safety and efficacy profile when compared to some traditional vaccine formulations [Vander Veen, et al. Anim Health Res Rev. 13 (1): 1-9. (2012)]. The RP platform has been used to encode pathogenic antigens and is the basis for several USDA-licensed vaccines for swine and poultry.

Unfortunately heretofore, pet owners were forced to choose between (i) nonadjuvanted FeLV vaccines that were believed to be safer, but found to be significantly less efficacious than killed, adjuvanted vaccines [see, Stuke et al., Vaccine 32:2599-2603 (2014); Patel et al., Clin Vaccine Immunol 22 (7): 798-808 (2015)] and (ii) the adjuvanted FeLV vaccines, which though more efficacious, are perceived by some to lead to injection-site sarcomas. Accordingly, there remains a present need for an improved, safe nonadjuvanted FeLV vaccine, which while not inducing feline injection-site sarcomas still protects vaccinates from the debilitating disease state caused by FeLV infection as efficaciously as its inactivated whole-virus adjuvanted vaccine counterpart.

The citation of any reference herein should not be construed as an admission that such reference is available as “prior art” to the instant application.

### SUMMARY OF THE INVENTION

Accordingly, the present invention provides vectors that encode one or more feline leukemia virus (FeLV) antigens. Such vectors can be used in immunogenic compositions comprising these vectors. The immunogenic compositions of the present invention may be used in vaccines. In one aspect of the present invention, a vaccine protects the vaccinated subject (e.g., mammal) against FeLV. In a particular embodiment of this type, the vaccinated subject is a feline. In a more particular embodiment, the vaccinated subject is a domestic cat. The present invention further provides combination vaccines for eliciting protective immunity against FeLV and other diseases, e.g., other feline infectious diseases. Methods of making and using the immunogenic compositions and vaccines of the present invention are also provided.

In specific embodiments, the vector is an alphavirus RNA replicon particle that encodes one or more antigens that originate from a feline pathogen. In particular embodiments, the feline pathogen is FeLV. In more particular embodiments, the alphavirus RNA replicon particles encode a FeLV glycoprotein (gp85). In related embodiments, the alphavirus RNA replicon particles encode an antigenic fragment of gp85. In a particular embodiment of this type, the antigenic fragment of gp85 is the FeLV glycoprotein gp70. In certain related embodiments, the antigenic fragment of gp85 is the FeLV glycoprotein gp45. In still more particular embodiments, the alphavirus RNA replicon particle is a Venezuelan Equine Encephalitis (VEE) alphavirus RNA replicon particle. In yet more specific embodiments the VEE alphavirus RNA replicon particle is a TC-83 VEE alphavirus RNA replicon particle. In other embodiments, the alphavirus RNA replicon particle is a Sindbis (SIN) alphavirus RNA replicon particle. In still other embodiments, the alphavirus RNA replicon particle is a Semliki Forest virus (SFV) alphavirus RNA replicon particle. In an alternative embodiment a naked DNA vector comprises a nucleic acid construct that encodes one or more antigens that originate from a feline pathogen. In particular embodiments of this type, the naked DNA vectors comprise a nucleic acid construct that encodes a FELV gp85, or antigenic fragment thereof.

In certain embodiments an alphavirus RNA replicon particle of the present invention encodes one or more FeLV antigens or antigenic fragments thereof. In particular embodiments of this type, the alphavirus RNA replicon particles encode two to four FeLV antigens or antigenic fragments thereof. In other embodiments, immunogenic compositions comprise alphavirus RNA replicon particles that encode one or more FeLV antigens or antigenic fragments thereof. In related embodiments, the immunogenic compositions comprise alphavirus RNA replicon particles encodes two to four FeLV antigens or antigenic fragments thereof. In particular embodiments of this type, the alphavirus RNA replicon particles encode an FeLV glycoprotein (gp85) or an antigenic fragment thereof. In more particular embodiments of this type, the antigenic fragment of gp85 is the FeLV glycoprotein gp70. In certain related embodiments, the antigenic fragment of gp85 is the FeLV glycoprotein gp45. In more particular embodiments, the immunogenic composition comprises alphavirus RNA replicon particles that are Venezuelan Equine Encephalitis (VEE) alphavirus RNA replicon particles. In yet more specific embodiments the VEE alphavirus RNA replicon particles are TC-83 VEE alphavirus RNA replicon particles.

In still other embodiments, the immunogenic composition comprises two or more sets of alphavirus RNA replicon particles. In particular embodiments of this type, one set of alphavirus RNA replicon particles encodes a particular antigen, whereas the other set of alphavirus RNA replicon particles encodes a second antigen. In a specific embodiment of this type the first set of alphavirus RNA replicon particles encode the FeLV antigen or an antigenic fragment thereof, and the second set of alphavirus RNA replicon particles encode a feline calicivirus (FCV) antigen or an antigenic fragment thereof. In certain embodiments of this type, the FCV antigen originates from a virulent systemic feline calicivirus (VS-FCV) isolate. In other embodiments the FCV antigen originates from a classical (F9-like) feline calicivirus isolate. In yet other embodiments, the second set of alphavirus RNA replicon particles encode two FCV antigens, one of which originates from a virulent systemic feline calicivirus isolate, whereas the other originates from a classical (F9-like) feline calicivirus isolate.

In yet other embodiments, the immunogenic composition comprises one set of alphavirus RNA replicon particles that encode a first antigen, another set of alphavirus RNA replicon particles that encode a second antigen, and a third set of alphavirus RNA replicon particles that encode a third antigen. In a particular embodiment of this type, the first set of alphavirus RNA replicon particles encode the FeLV antigen or an antigenic fragment thereof, the second set of alphavirus RNA replicon particles encode a feline calicivirus (FCV) antigen which originates from a virulent systemic feline calicivirus or an antigenic fragment thereof, and the third set of alphavirus RNA replicon particles encode a feline calicivirus (FCV) antigen which originates from a classical (F9-like) feline calicivirus or an antigenic fragment thereof.

Accordingly, in particular embodiments in which the immunogenic compositions comprise multiple sets (e.g., 2-10) of alphavirus RNA replicon particles, in which the first set of alphavirus RNA replicon particles encode the FeLV antigen or an antigenic fragment thereof, the one or more other sets of alphavirus RNA replicon particles can encode one or more non-FeLV antigens. In certain embodiments of this type, the non-FeLV antigen is a protein antigen that originates from feline herpesvirus (FHV). In other embodiments, the non-FeLV antigen is a protein antigen that originates from feline calicivirus (FCV). In yet other embodiments, the non-FeLV antigen is a protein antigen that originates from feline pneumovirus (FPN). In still other embodiments, the non-FeLV antigen is a protein antigen that originates from feline parvovirus (FPV). In yet other embodiments, the non-FeLV antigen is a protein antigen that originates from feline infectious peritonitis virus (FIPV). In still other embodiments, the non-FeLV antigen is a protein antigen that originates from feline immunodeficiency virus. In still other embodiments, the non-FeLV antigen is a protein antigen that originates from borna disease virus (BDV). In yet other embodiments, the non-FeLV antigen is a protein antigen that originates from feline influenza virus. In still other embodiments, the non-FeLV antigen is a protein antigen that originates from feline panleukopenia virus (FPLV). In yet other embodiments the non-FeLV antigen is a protein antigen that originates from feline coronavirus (FCoV). In still other embodiments the non-FeLV antigen is a protein antigen that originates from feline rhinotracheitis virus (FVR). In yet other embodiments the non-FeLV antigen is a protein antigen that originates from Chlamydophila felis.

The present invention also includes all of the nucleic acid constructs of the present invention including synthetic messenger RNA, RNA replicons, as well as all of the alphavirus RNA replicon particles of the present invention, the naked DNA vectors, and the immunogenic compositions and/or vaccines that comprise the nucleic acid constructs (e.g., synthetic messenger RNA, RNA replicons), the alphavirus RNA replicon particles, and/or the naked DNA vectors of the present invention.

In particular embodiments, a nucleic acid construct of the present invention encodes one or more FeLV antigens or antigenic fragments thereof. In related embodiments of this type, the nucleic acid construct encodes two to four FeLV antigens or antigenic fragments thereof. In other embodiments, alphavirus RNA replicon particles comprise a nucleic acid construct that encodes one or more FeLV antigens or antigenic fragments thereof. In particular embodiments, alphavirus RNA replicon particles comprise a nucleic acid construct that encodes two to four FeLV antigens or antigenic fragments thereof.

In still other embodiments, the immunogenic compositions comprise alphavirus RNA replicon particles that comprise a nucleic acid construct that encodes two to four FeLV antigens or antigenic fragments thereof. In particular embodiments of this type, the alphavirus RNA replicon particles comprise a nucleic acid construct encoding an FeLV glycoprotein (gp85) or an antigenic fragment thereof. In a particular embodiment of this type, the antigenic fragment of gp85 is the FeLV glycoprotein gp70. In other related embodiments, the antigenic fragment of gp85 is the FeLV glycoprotein gp45. In more particular embodiments, the immunogenic composition comprises alphavirus RNA replicon particles that are Venezuelan Equine Encephalitis (VEE) alphavirus RNA replicon particles. In yet more specific embodiments the VEE alphavirus RNA replicon particles are TC-83 VEE alphavirus RNA replicon particles.

In yet other embodiments, the immunogenic composition comprises two or more sets of alphavirus RNA replicon particles. In particular embodiments of this type, one set of alphavirus RNA replicon particles comprises a first nucleic acid construct, whereas the other set of alphavirus RNA replicon particles comprise a second nucleic acid construct. In a specific embodiment of this type the first nucleic acid construct encodes the FeLV antigen or an antigenic fragment thereof, and the second nucleic acid construct encodes a feline calicivirus (FCV) antigen or an antigenic fragment thereof. In certain embodiments of this type, the FCV antigen originates from a virulent systemic feline calicivirus (VS-FCV) isolate. In other embodiments the FCV antigen originates from a classical (F9-like) feline calicivirus isolate. In yet other embodiments, the second nucleic acid construct encodes two FCV antigens, one of which originates from a virulent systemic feline calicivirus isolate, whereas the other originates from a classical (F9-like) feline calicivirus isolate.

In still other embodiments, the immunogenic composition comprises one set of alphavirus RNA replicon particles that comprise a first nucleic acid construct, another set of alphavirus RNA replicon particles that comprise a second nucleic acid construct, and a third set of alphavirus RNA replicon particles that comprise a third nucleic acid construct. In a particular embodiment of this type, the first nucleic acid construct encodes the FeLV antigen or an antigenic fragment thereof, the second nucleic acid construct encodes a feline calicivirus (FCV) antigen which originates from a virulent systemic feline calicivirus or an antigenic fragment thereof, and the third nucleic acid construct encodes a feline calicivirus (FCV) antigen which originates from a classical (F9-like) feline calicivirus or an antigenic fragment thereof.

In yet other embodiments, the immunogenic composition comprises one set of alphavirus RNA replicon particles that comprise a first nucleic acid construct, another set of alphavirus RNA replicon particles that comprise a second nucleic acid construct, a third set of alphavirus RNA replicon particles that comprise a third nucleic acid construct, and a fourth set of alphavirus RNA replicon particles that comprise a fourth nucleic acid construct. In yet other embodiments, the immunogenic composition comprises a set of alphavirus RNA replicon particles that comprise a first nucleic acid construct, another set of alphavirus RNA replicon particles that comprise a second nucleic acid construct, a third set of alphavirus RNA replicon particles that comprise a third nucleic acid construct, a fourth set of alphavirus RNA replicon particles that comprise a fourth nucleic acid construct, and a fifth set of alphavirus RNA replicon particles that comprise a fifth nucleic acid construct. In such embodiments, the nucleotide sequences of the first nucleic acid construct, the second nucleic acid construct, third nucleic acid construct, the fourth nucleic acid construct, and the fifth nucleic acid construct are all different.

Accordingly, an immunogenic composition of the present invention can contain alphavirus RNA replicon particles that comprise a nucleic acid construct that encodes at least one non-FeLV antigen for eliciting protective immunity to a non-FeLV pathogen. In particular embodiments of this type, the non-FeLV antigen is a protein antigen that originates from feline herpesvirus (FHV). In other embodiments, the non-FeLV antigen is a protein antigen that originates from feline calicivirus (FCV). In yet other embodiments, the non-FeLV antigen is a protein antigen that originates from feline pneumovirus (FPN). In still other embodiments, the non-FeLV antigen is a protein antigen that originates from feline parvovirus (FPV). In yet other embodiments, the non-FeLV antigen is a protein antigen that originates from feline infectious peritonitis virus (FIPV). In still other embodiments, the non-FeLV antigen is a protein antigen that originates from feline immunodeficiency virus. In still other embodiments, the non-FeLV antigen is a protein antigen that originates from borna disease virus (BDV). In yet other embodiments, the non-FeLV antigen is a protein antigen that originates from feline influenza virus. In still other embodiments, the non-FeLV antigen is a protein antigen that originates from feline panleukopenia virus (FPLV). In yet other embodiments the non-FeLV antigen is a protein antigen that originates from feline coronavirus (FCoV). In still other embodiments the non-FeLV antigen is a protein antigen that originates from feline rhinotracheitis virus (FVR). In still other embodiments the non-FeLV antigen is a protein antigen that originates from Chlamydophila felis.

The present invention further provides combination immunogenic compositions and/or vaccines (multivalent vaccines) that include alphavirus RNA replicon particles that encode an antigen or antigenic fragment thereof originating from FeLV together with one or more modified live (e.g., attenuated) or killed feline pathogens. In particular embodiments, the immunogenic compositions comprise a modified live or killed Chlamydophila felis combined with alphavirus RNA replicon particles that encode an antigen or antigenic fragment thereof originating from FeLV. In other embodiments, the immunogenic compositions comprise a modified live or killed feline rhinotracheitis Virus (FVR) combined with alphavirus RNA replicon particles that encode an antigen or antigenic fragment thereof originating from FELV. In still other embodiments, the immunogenic compositions comprise a modified live or killed feline calicivirus (FCV) combined with alphavirus RNA replicon particles that encode an antigen or antigenic fragment thereof originating from FELV. In yet other embodiments, the immunogenic compositions comprise a modified live or killed feline panleukopenia virus (FPL) combined with alphavirus RNA replicon particles that encode an antigen or antigenic fragment thereof originating from FeLV. In still other embodiments, the immunogenic compositions comprise a modified live or killed Chlamydophila felis, a modified live or killed FVR, a modified live or killed FCV, a modified live or killed FPL, and alphavirus RNA replicon particles that encode an antigen or antigenic fragment thereof originating from FELV. In particular embodiments of this type, the feline antigen of the FeLV is the FeLV viral glycoprotein (gp85). In certain embodiments, vaccines comprise an immunologically effective amount of one or more of these immunogenic compositions.

In particular embodiments the FeLV antigen is the FeLV glycoprotein (gp85). In specific embodiments of this type, the FeLV glycoprotein gp85 comprises an amino acid sequence comprising 95% identity or more with the amino acid sequence of SEQ ID NO: 2. In more specific embodiments of this type, the FeLV glycoprotein (gp85) comprises the amino acid sequence of SEQ ID NO: 2. In even more specific embodiments of this type the FLV glycoprotein (gp85) is encoded by the nucleotide sequence of SEQ ID NO: 1 or SEQ ID NO: 10. In related embodiments, the FeLV glycoprotein gp70 comprises an amino acid sequence comprising 95% identity or more with the amino acid sequence of SEQ ID NO: 4. In more specific embodiments of this type, the FeLV glycoprotein (gp85) comprises the amino acid sequence of SEQ ID NO: 4. In even more specific embodiments of this type the FcLV glycoprotein (gp70) is encoded by the nucleotide sequence of SEQ ID NO: 3 or SEQ ID NO: 11.

The present invention further comprises vaccines and multivalent vaccines comprising the immunogenic compositions of the present invention. In particular embodiments, the vaccines are nonadjuvanted vaccine. In certain embodiments, the vaccine aids in the prevention of disease due to FeLV. In related embodiments, antibodies are induced in a feline subject when the feline is immunized with the vaccine.

The present invention also provides methods of immunizing a feline against a feline pathogen, e.g., FeLV, comprising administering to the feline an immunologically effective amount of a vaccine or multivalent of the present invention. In particular embodiments the vaccine is administered via intramuscular injection. In alternative embodiments the vaccine is administered via subcutaneous injection. In other embodiments the vaccine is administered via intravenous injection. In still other embodiments the vaccine is administered via intradermal injection. In yet other embodiments the vaccine is administered via oral administration. In still other embodiments the vaccine is administered via intranasal administration. In specific embodiments, the feline is a domestic cat.

The vaccines and multivalent vaccines of the present invention can be administered as a primer vaccine and/or as a booster vaccine. In specific embodiments, a vaccine of the present invention is administered as a one shot vaccine (one dose), without requiring subsequent administrations. In certain embodiments, in the case of the administration of both a primer vaccine and a booster vaccine, the primer vaccine and the booster vaccine can be administered by the identical route. In certain embodiments of this type, the primer vaccine and the booster vaccine are both administered by subcutaneous injection. In alternative embodiments, in the case of the administration of both a primer vaccine and a booster vaccine, the administration of the primer vaccine can be performed by one route and the booster vaccine by another route. In certain embodiments of this type, the primer vaccine can be administered by subcutaneous injection and the booster vaccine can be administered orally.

The invention further provides for a method of immunizing a feline against FeLV comprising injecting the feline with an immunologically effective amount of the above described inventive vaccines. In particular embodiments the vaccines can include from about 1×10⁴ to about 1×10¹⁰ RPs or higher, for example. In more particular embodiments the vaccines can include from about 1×10⁵ to about 1×10⁹ RPs. In even more particular embodiments the vaccines can include from about 1×10⁶ to about 1×10⁸ RPs. In particular embodiments the feline is a domestic cat.

In particular embodiments the vaccines of the present invention are administered in 0.05 mL to 3 mL doses. In more particular embodiments the dose administered is 0.1 mL to 2 mLs. In still more particular embodiments the dose administered is 0.2 mL to 1.5 mLs. In even more particular embodiments the dose administered is 0.3 to 1.0 mLs. In still more particular embodiments the dose administered is 0.4 mL to 0.8 mLs. In yet more particular embodiments the dose administered is 0.5 mL to 1.5 mLs.

These and other aspects of the present invention will be better appreciated by reference to the following Detailed Description.

### DETAILED DESCRIPTION OF THE INVENTION

The present invention provides an improved, safe nonadjuvanted FeLV vaccine. In one aspect, the vaccines of the present invention do not induce feline injection-site sarcomas, yet still provide protection to the vaccinates from the debilitating disease state caused by FeLV infection as efficaciously as an inactivated whole-virus adjuvanted vaccine.

Accordingly, the vaccine compositions of the present invention include an immunologically effective amount of a vector encoding an antigen from one or more strains of feline leukemia virus that aids in eliciting protective immunity in the recipient vaccinated animal. Furthermore, the present invention provides new immunologic compositions to improve the reliability of vaccination to aid in the reduction of antigenemia in a feline infected by FeLV and to thereby yield a transient antigenemia and/or lead to the elimination of the infection. In a particular aspect of the present invention, the vaccines comprise an alphavirus RNA replicon particle (RP) encoding the FeLV viral glycoprotein (gp85). In more specific embodiments, the vaccines comprise alphavirus RNA replicon particles (RPs) that comprise the capsid protein and glycoproteins of Venezuelan Equine Encephalitis Virus (VEE) and encode the FeLV viral glycoprotein (gp85) and/or an antigenic fragment thereof (e.g., gp70 or gp45). In even more specific embodiments, the vaccines comprise alphavirus RNA replicon particles (RPs) that comprise the capsid protein and glycoproteins of the avirulent TC-83 strain of VEE and encode the FeLV viral glycoprotein (gp85) and/or an antigenic fragment thereof (e.g., gp70 or gp45).

In another aspect of the present invention, the vaccines comprise naked DNA vectors that encode the FeLV viral glycoprotein (gp85) and/or an antigenic fragment thereof (e.g., gp70 or gp45). The vaccines of the present invention can be administered to a feline in the absence of an adjuvant and still effectively aid in the protection of the vaccinated feline against FeLV.

In order to more fully appreciate the invention, the following definitions are provided.

The use of singular terms for convenience in description is in no way intended to be so limiting. Thus, for example, reference to a composition comprising “a polypeptide” includes reference to one or more of such polypeptides. In addition, reference to an “alphavirus RNA replicon particle” includes reference to a plurality of such alphavirus RNA replicon particles, unless otherwise indicated.

As used herein the term “approximately” is used interchangeably with the term “about” and signifies that a value is within fifty percent of the indicated value i.e., a composition containing “approximately” 1×10⁸ alphavirus RNA replicon particles per milliliter contains from 0.5×10⁸ to 1.5×10⁸ alphavirus RNA replicon particles per milliliter.

As used herein, the term “feline” refers to any member of the Felidae family. Domestic cats, pure-bred and/or mongrel companion cats, and wild or feral cats are all felines.

As used herein, the term “replicon” refers to a modified RNA viral genome that lacks one or more elements (e.g., coding sequences for structural proteins) that if they were present, would enable the successful propagation of the parental virus in cell cultures or animal hosts. In suitable cellular contexts, the replicon will amplify itself and may produce one or more sub-genomic RNA species.

As used herein, the term “alphavirus RNA replicon particle”, abbreviated “RP”, is an alphavirus-derived RNA replicon packaged in structural proteins, e.g., the capsid and glycoproteins, which also are derived from an alphavirus, e.g., as described by Pushko et al., [Virology 239 (2): 389-401 (1997)]. An RP cannot propagate in cell cultures or animal hosts (without a helper plasmid or analogous component), because the replicon does not encode the alphavirus structural components (e.g., capsid and glycoproteins).

The term “non-FeLV”, is used to modify terms such as pathogen, and/or antigen (or immunogen) to signify that the respective pathogen, and/or antigen (or immunogen) is neither an FeLV pathogen nor a FeLV antigen (or immunogen) and that a non-FeLV protein antigen (or immunogen) does not originate from an FeLV.

The terms “originate from”, “originates from” and “originating from” are used interchangeably with respect to a given protein antigen and the pathogen or strain of that pathogen that naturally encodes it, and as used herein signify that the unmodified and/or truncated amino acid sequence of that given protein antigen is encoded by that pathogen or strain of that pathogen. The coding sequence within a nucleic acid construct of the present invention for a protein antigen originating from a pathogen may have been genetically manipulated so as to result in a modification and/or truncation of the amino acid sequence of the expressed protein antigen relative to the corresponding sequence of that protein antigen in the pathogen or strain of pathogen (including naturally attenuated strains) it originates from.

As used herein, the terms “protecting”, or “providing protection to”, or “eliciting protective immunity to”, “aids in prevention of disease”, and “aids in the protection” do not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, a “vaccine” is a composition that is suitable for application to an animal, e.g., feline (including, in certain embodiments, humans, while in other embodiments being specifically not for humans) comprising one or more antigens typically combined with a pharmaceutically acceptable carrier such as a liquid containing water, which upon administration to the animal induces an immune response strong enough to minimally aid in the protection from a disease arising from an infection with a wild-type micro-organism, i.e., strong enough for aiding in the prevention of the disease, and/or preventing, ameliorating or curing the disease.

As used herein, a multivalent vaccine is a vaccine that comprises two or more different antigens. In a particular embodiment of this type, the multivalent vaccine stimulates the immune system of the recipient against two or more different pathogens.

The terms “adjuvant” and “immune stimulant” are used interchangeably herein, and are defined as one or more substances that cause stimulation of the immune system. In this context, an adjuvant is used to enhance an immune response to one or more vaccine antigens/isolates. Accordingly, “adjuvants” are agents that nonspecifically increase an immune response to a particular antigen, thus reducing the quantity of antigen necessary in any given vaccine, and/or the frequency of injection necessary in order to generate an adequate immune response to the antigen of interest. In this context, an adjuvant is used to enhance an immune response to one or more vaccine antigens/isolates. The American Association of Feline Practitioners Feline Vaccination Guidelines suggest the use of nonadjuvanted FeLV vaccines [AAFP Feline Advisory Panel, 15:785-808 (2013)].

As used herein, a “nonadjuvanted vaccine” is a vaccine or a multivalent vaccine that does not contain an adjuvant.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient animal, e.g., feline.

Parenteral administration” includes subcutaneous injections, submucosal injections, intravenous injections, intramuscular injections, intradermal injections, and infusion.

As used herein the term “antigenic fragment” in regard to a particular protein (e.g., a protein antigen) is a fragment of that protein that is antigenic, i.e., capable of specifically interacting with an antigen recognition molecule of the immune system, such as an immunoglobulin (antibody) or T cell antigen receptor. For example, an antigenic fragment of an FeLV viral glycoprotein (gp85) is a fragment of the gp85 protein that is antigenic. Preferably, an antigenic fragment of the present invention is immunodominant for antibody and/or T cell receptor recognition. In particular embodiments, an antigenic fragment with respect to a given protein antigen is a fragment of that protein that retains at least 25% of the antigenicity of the full length protein. In preferred embodiments an antigenic fragment retains at least 50% of the antigenicity of the full length protein. In more preferred embodiments, an antigenic fragment retains at least 75% of the antigenicity of the full length protein. Antigenic fragments can be as small as 20 amino acids or at the other extreme, be large fragments that are missing as little as a single amino acid from the full-length protein. In particular embodiments the antigenic fragment comprises 25 to 150 amino acid residues. In other embodiments, the antigenic fragment comprises 50 to 250 amino acid residues. The gp45 glycoprotein and the gp70 glycoprotein are antigenic fragments of the gp85 glycoprotein.

As used herein one amino acid sequence is 100% “identical” or has 100% “identity” to a second amino acid sequence when the amino acid residues of both sequences are identical. Accordingly, an amino acid sequence is 50% “identical” to a second amino acid sequence when 50% of the amino acid residues of the two amino acid sequences are identical.

The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

As used herein, nucleotide and amino acid sequence percent identity can be determined using C, Mac Vector (Mac Vector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

As used herein, the term “inactivated” microorganism is used interchangeably with the term “killed” microorganism. For the purposes of this invention, an “inactivated” microorganism is an organism which is capable of eliciting an immune response in an animal, but is not capable of infecting the animal. An antigen of the present invention (e.g., an inactivated feline panleukopenia virus) may be inactivated by an agent selected from the group consisting of binary ethyleneimine, formalin, beta-propiolactone, thimerosal, or heat. In a particular embodiment, inactivated feline calicivirus isolates combined with an RP of the present invention are inactivated by binary ethyleneimine.

The alphavirus RNA replicon particles of the present invention may be lyophilized and rehydrated with a sterile water diluent. On the other hand, when the alphavirus RNA replicon particles are stored separately, but intend to be mixed with other vaccine components prior to administration, the alphavirus RNA replicon particles can be stored in the stabilizing solution of those components, e.g., a high sucrose solution.

A vaccine of the present invention can be readily administered by any standard route including intravenous, intramuscular, subcutaneous, oral, intranasal, intradermal, and/or intraperitoneal vaccination. The skilled artisan will appreciate that the vaccine composition is preferably formulated appropriately for each type of recipient animal and route of administration.

Thus, the present invention also provides methods of immunizing a feline against FeLV and/or other feline pathogens. One such method comprises injecting a feline with an immunologically effective amount of a vaccine of the present invention, so that the feline produces appropriate FeLV antibodies.

### Multivalent Vaccines:

The present invention also provides multivalent vaccines. For example, the coding sequence of a protein antigen or antigenic fragment thereof, or combination of such coding sequences of protein antigens useful in a feline vaccine can be added to an alphavirus RNA replicon particle (RP) or combined in the same RP as one that encodes a feline antigen of the FeLV [e.g., the FeLV viral glycoprotein (gp85)] in the vaccine. Accordingly, such multivalent vaccines are included in the present invention.

Examples of pathogens that one or more of such protein antigens can originate from include feline rhinotracheitis Virus (FVR), feline calicivirus (FCV), feline panleukopenia Virus (FPL) feline herpesvirus (FHV), other FeLV strains, feline parvovirus (FPV), feline infectious peritonitis virus (FIPV), feline immunodeficiency virus, borna disease virus (BDV), rabies virus, feline influenza virus, canine influenza virus, avian influenza, canine pneumovirus, feline pneumovirus, Chlamydophila felis (FKA Chlamydia psittaci), Bordetella bronchiseptica, and Bartonella spp. (e.g., B. henselae). In particular embodiments, a coding sequence for a capsid protein or analogous protein from one or more of these feline or canine pathogens can be inserted into the same RP as the FeLV antigen. Alternatively, or in combination therewith, a coding sequence for a capsid protein or analogous protein from one or more of these feline or canine pathogens can be inserted into one or more other RPs, which can be combined with the RP that encodes the FeLV antigen in a vaccine.

In addition, an alphavirus RNA replicon particle (RP) that encodes a feline antigen of the FeLV [e.g., the FeLV viral glycoprotein (gp85)] can be added together with one or more other live, attenuated virus isolates such as a live attenuated other FCV strain, a live attenuated feline herpesvirus and/or a live attenuated feline parvovirus and/or a live, attenuated feline leukemia virus, and/or a live, attenuated feline infectious peritonitis virus and/or a live, attenuated feline immunodeficiency virus and/or a live, attenuated borna disease virus and/or a live, attenuated rabies virus, and/or a live, attenuated feline influenza virus and/or a live, attenuated canine influenza virus, and/or a live, attenuated avian influenza, and/or a live, attenuated canine pneumovirus, and/or a live, attenuated feline pneumovirus. In addition, a live, attenuated Chlamydophila felis, and/or a live, attenuated Bordetella bronchiseptica and/or a live, attenuated Bartonella spp. (e.g., B. henselae) can also be included in such multivalent vaccines.

Furthermore, an alphavirus RNA replicon particle (RP) that encodes a feline antigen of the FeLV [e.g., the FeLV viral glycoprotein (gp85)] can be added together with one or more other killed virus isolates such as a killed FCV strain, and/or a killed feline herpesvirus and/or a killed feline parvovirus and/or a killed feline leukemia virus, and/or a killed feline infectious peritonitis virus and/or a killed feline immunodeficiency virus and/or a killed borna disease virus and/or a killed rabies virus, and/or a killed feline influenza virus and/or a killed canine influenza virus, and/or a killed avian influenza virus, and/or a killed canine pneumovirus, and/or a killed feline pneumovirus. In addition, bacterins of Chlamydophila felis, and/or Bordetella bronchiseptica and/or Bartonella spp. (e.g., B. henselae) can also be included in such multivalent vaccines.

It is also to be understood that this invention is not limited to the particular configurations, process steps, and materials disclosed herein as such configurations, process steps, and materials may vary somewhat.

It is also to be understood that the terminology employed herein is used for the purpose of describing particular embodiments only and is not intended to be limiting, since the scope of the present invention will be limited only by the appended claims and equivalents thereof.

SEQUENCE TABLE

| SEQ ID NO: | Description | Type |
|---|---|---|
| 1 | FeLV viral | nucleic |
| | glycoprotein (gp85) | acid DNA |
| 2 | FeLV viral | amino acid |
| | glycoprotein (gp85) | |
| 3 | FeLV viral | nucleic |
| | glycoprotein (gp70) | acid DNA |
| 4 | FeLV viral | amino acid |
| | glycoprotein (gp70) | |
| 5 | Feline Calicivirus | nucleic |
| | (VS-FCV) | acid DNA |
| 6 | Feline Calicivirus | amino acid |
| | (VS-FCV) | |
| 7 | Feline Calicivirus | nucleic |
| | (F9-like) | acid DNA |
| 8 | Feline Calicivirus | amino acid |
| | (F9-like) | |
| 9 | GGCGCGCCGCACC | nucleic acid |
| 10 | FeLV viral | nucleic |
| | glycoprotein (gp85) | acid RNA |
| 11 | FeLV viral | nucleic |
| | glycoprotein (gp70) | acid RNA |
| 12 | Feline Calicivirus | nucleic |
| | (VS-FCV) | acid RNA |
| 13 | Feline Calicivirus | nucleic |
| | (F9-like) | acid RNA |
| | TTAATTAA | nucleic acid |

SEQUENCES

Feline Leukemia Virus envelope glycoprotein (gp85)
SEQ ID NO: 1
atggagtcaccaacacaccctaaaccttctaaagacaaaaccctctcgtggaatctcgccttccttgt
gggcatcctgttcacaatcgacatcggcatggccaacccttcgccgcatcagatctacaatgtgacat
gggtcattactaatgtgcagacaaacacccaggcaaatgctacttctatgcttggtactctgactgat
gcttatccaaccctgcacgtcgacctttgcgatctcgtcggtgacacatgggagcccatcgtgctgaa
tccaactaatgtcaaacatggtgccaggtattcttctagcaaatacgggtgtaagaccactgatcgga
agaaacagcaacaaacctacccattctacgtgtgcccgggtcacgcaccgtccctgggtccgaaggga
acacattgtgggggagcccaagacggtttttgcgctgcttggggttgtgaaacaaccggagaagcctg
gtggaagcctacctcatcttgggactacattactgtgaaaagaggctctagccaggataacagctgcg
aaggaaagtgtaatcccctggtgcttcaattcacccagaaaggccggcaggcatcatgggatggaccg
aaaatgtggggacttagactctatcgcaccggatacgaccccatcgctctgtttactgtgtcacgcca
agtctccaccattactccgccacaggccatggggccgaatctggtcctccccgatcagaagccaccct
cacggcaaagtcaaaccggctcaaaagtggccacccaacggccccagacaaatgagtccgcacctagg
tcagtggcacctacaacaatgggtccaaagcggatcggaaccggagacaggctcattaacctcgtgca
agggacttatctggcccttaacgctactgaccccaacaagaccaaggattgctggctctgccttgtga
gcagacctccttactatgaggggatcgccattctcggaaactactcaaatcagaccaacccccctccg
tcgtgtctgagcaccccccagcacaagcttactatttcagaagtcagtggacagggaatgtgcatcgg
aaccgtgccaaagactcatcaagccctttgcaacaaaactcaacaagggcacactggagctcattatc
tcgccgcacctaacgggacctactgggcttgcaatactggattgaccccgtgtatctctatggccgtg
ctgaattggacttccgacttctgcgtgcttattgagctttggcctagagtgacataccatcagcctga
gtacgtctatacccatttcgccaaggcagtcagattccggcgggagcctatctccctgactgtggcct
tgatgctcggtggactgacagtgggaggaattgcagctggagtcggaactggaaccaaggccctgctc
gaaactgctcagttccggcagctgcagatggccatgcacactgacatccaggctctggaggaatcaat
ttcagcccttgagaaaagcttgacctcgctgtctgaagtggtcctccaaaacaggcgcggtttggaca
tcctgttccttcaagagggtggtctgtgcgccgctctcaaggaggaatgctgtttctacgctgaccat
accgggctggtgcgcgataacatggcaaagctgcgggaacgcttgaaacagaggcagcaactgttcga
ctctcagcagggatggttcgagggctggtttaacaagagcccatggtttaccactctgatctcttcaa
tcatgggtccactgctcatcctgcttctgattcttctcttcggaccgtgtattctcaacaggctggtg
cagtttgtcaaggacagaatctcggtggtccaggccctgattcttactcagcagtatcagcagattaa
gcagtacgaccccgatcggccttga
Feline Leukemia Virus envelope glycoprotein (gp85)
SEQ ID NO: 2
MESPTHPKPSKDKTLSWNLAFLVGILFTIDIGMANPSPHQIYNVTWVITNVQTNTQANATSMLGTLTD
AYPTLHVDLCDLVGDTWEPIVLNPTNVKHGARYSSSKYGCKTTDRKKQQQTYPFYVCPGHAPSLGPKG
THCGGAQDGFCAAWGCETTGEAWWKPTSSWDYITVKRGSSQDNSCEGKCNPLVLQFTQKGRQASWDGP
KMWGLRLYRTGYDPIALFTVSRQVSTITPPQAMGPNLVLPDQKPPSRQSQTGSKVATQRPQTNESAPR
SVAPTTMGPKRIGTGDRLINLVQGTYLALNATDPNKTKDCWLCLVSRPPYYEGIAILGNYSNQTNPPP
SCLSTPQHKLTISEVSGQGMCIGTVPKTHQALCNKTQQGHTGAHYLAAPNGTYWACNTGLTPCISMAV
LNWTSDFCVLIELWPRVTYHQPEYVYTHFAKAVRFRREPISLTVALMLGGLTVGGIAAGVGTGTKALL
ETAQFRQLQMAMHTDIQALEESISALEKSLTSLSEVVLQNRRGLDILFLQEGGLCAALKEECCFYADH
TGLVRDNMAKLRERLKQRQQLFDSQQGWFEGWFNKSPWFTTLISSIMGPLLILLLILLFGPCILNRLV
QFVKDRISVVQALILTQQYQQIKQYDPDRP*
Feline Leukemia Virus envelope glycoprotein (gp85)
SEQ ID NO: 10
auggagucaccaacacacccuaaaccuucuaaagacaaaacccucucguggaaucucgccuuccuugu
gggcauccuguucacaaucgacaucggcauggccaacccuucgccgcaucagaucuacaaugugacau
gggucauuacuaaugugcagacaaacacccaggcaaaugcuacuucuaugcuugguacucugacugau
gcuuauccaacccugcacgucgaccuuugcgaucucgucggugacacaugggagcccaucgugcugaa
uccaacuaaugucaaacauggugccagguauucuucuagcaaauacggguguaagaccacugaucgga
agaaacagcaacaaaccuacccauucuacgugugcccgggucacgcaccgucccuggguccgaaggga
acacauugugggggagcccaagacgguuuuugcgcugcuugggguugugaaacaaccggagaagccug
guggaagccuaccucaucuugggacuacauuacugugaaaagaggcucuagccaggauaacagcugcg
aaggaaaguguaauccccuggugcuucaauucacccagaaaggccggcaggcaucaugggauggaccg
aaaauguggggacuuagacucuaucgcaccggauacgaccccaucgcucuguuuacugugucacgcca
agucuccaccauuacuccgccacaggccauggggccgaaucugguccuccccgaucagaagccacccu
cacggcaaagucaaaccggcucaaaaguggccacccaacggccccagacaaaugaguccgcaccuagg
ucaguggcaccuacaacaauggguccaaagcggaucggaaccggagacaggcucauuaaccucgugca
agggacuuaucuggcccuuaacgcuacugaccccaacaagaccaaggauugcuggcucugccuuguga
gcagaccuccuuacuaugaggggaucgccauucucggaaacuacucaaaucagaccaaccccccuccg
ucgugucugagcaccccccagcacaagcuuacuauuucagaagucaguggacagggaaugugcaucgg
aaccgugccaaagacucaucaagcccuuugcaacaaaacucaacaagggcacacuggagcucauuauc
ucgccgcaccuaacgggaccuacugggcuugcaauacuggauugaccccguguaucucuauggccgug
cugaauuggacuuccgacuucugcgugcuuauugagcuuuggccuagagugacauaccaucagccuga
guacgucuauacccauuucgccaaggcagucagauuccggcgggagccuaucucccugacuguggccu
ugaugcucgguggacugacagugggaggaauugcagcuggagucggaacuggaaccaaggcccugcuc
gaaacugcucaguuccggcagcugcagauggccaugcacacugacauccaggcucuggaggaaucaau
uucagcccuugagaaaagcuugaccucgcugucugaagugguccuccaaaacaggcgcgguuuggaca
uccuguuccuucaagaggguggucugugcgccgcucucaaggaggaaugcuguuucuacgcugaccau
accgggcuggugcgcgauaacauggcaaagcugcgggaacgcuugaaacagaggcagcaacuguucga
cucucagcagggaugguucgagggcugguuuaacaagagcccaugguuuaccacucugaucucuucaa
ucauggguccacugcucauccugcuucugauucuucucuucggaccguguauucucaacaggcuggug
caguuugucaaggacagaaucucggugguccaggcccugauucuuacucagcaguaucagcagauuaa
gcaguacgaccccgaucggccuuga
Feline Leukemia Virus envelope glycoprotein (gp70)
SEQ ID NO: 3
aatcctagtccacaccaaatatataatgtaacttgggtaataaccaatgtacaaactaacacc
caagctaacgccacctctatgttaggaaccttaaccgatgcctaccctaccctacatgttgac
ttatgtgacctagtgggagacacctgggaacctatagtcctaaacccaaccaatgtaaaacac
ggggcacgttactcctcctcaaaatatggatgtaaaactacagatagaaaaaaacagcaacag
acataccccttttacgtctgccccggacatgccccctcgttggggccaaagggaacacattgt
ggaggggcacaagatgggttttgtgccgcatggggatgtgagaccaccggagaagcttggtgg
aagcccacctcctcatgggactatatcacagtaaaaagagggagtagtcaggacaatagctgt
gagggaaaatgcaaccccctggttttgcagttcacccagaagggaagacaagcctcttgggac
ggacctaagatgtggggattgcgactataccgtacaggatatgaccctatcgctttattcacg
gtgtcccggcaggtatcaaccattacgccgcctcaggcaatgggaccaaacctagtcttacct
gatcaaaaacccccatcccgacaatctcaaacagggtccaaagtggcgacccagaggccccaa
acgaatgaaagcgccccaaggtctgttgcccccaccaccatgggtcccaaacggattgggacc
ggagataggttaataaatttagtacaagggacatacctagccttaaatgccaccgaccccaac
aaaactaaagactgttggctctgcctggtttctcgaccaccctattacgaagggattgcaatc
ttaggtaactacagcaaccaaacaaacccccccccatcctgcctatctactccgcaacacaaa
ctaactatatctgaagtatcagggcaaggaatgtgcatagggactgttcctaaaacccaccag
gctttgtgcaataagacacaacagggacatacaggggcgcactatctagccgcccccaacggc
acctattgggcctgtaacactggactcaccccatgcatttccatggcggtgctcaattggacc
tctgatttttgtgtcttaatcgaattatggcccagagtgacttaccatcaacccgaatatgtg
tacacacattttgccaaagctgtcaggttccgaaga
Feline Leukemia Virus envelope glycoprotein (gp70)
SEQ ID NO: 4
NPSPHQIYNVTWVITNVQTNTQANATSMLGTLTDAYPTLHVDLCDLVGDTWEPIVLNPTNVKH
GARYSSSKYGCKTTDRKKQQQTYPFYVCPGHAPSLGPKGTHCGGAQDGFCAAWGCETTGEAWW
KPTSSWDYITVKRGSSQDNSCEGKCNPLVLQFTQKGRQASWDGPKMWGLRLYRTGYDPIALFT
VSRQVSTITPPQAMGPNLVLPDQKPPSRQSQTGSKVATQRPQTNESAPRSVAPTTMGPKRIGT
GDRLINLVQGTYLALNATDPNKTKDCWLCLVSRPPYYEGIAILGNYSNQTNPPPSCLSTPQHK
LTISEVSGQGMCIGTVPKTHQALCNKTQQGHTGAHYLAAPNGTYWACNTGLTPCISMAVLNWT
SDFCVLIELWPRVTYHQPEYVYTHFAKAVRFRR
Feline Leukemia Virus envelope glycoprotein (gp70)
SEQ ID NO: 11
aauccuaguccacaccaaauauauaauguaacuuggguaauaaccaauguacaaacuaacacc
caagcuaacgccaccucuauguuaggaaccuuaaccgaugccuacccuacccuacauguugac
uuaugugaccuagugggagacaccugggaaccuauaguccuaaacccaaccaauguaaaacac
ggggcacguuacuccuccucaaaauauggauguaaaacuacagauagaaaaaaacagcaacag
acauaccccuuuuacgucugccccggacaugcccccucguuggggccaaagggaacacauugu
ggaggggcacaagauggguuuugugccgcauggggaugugagaccaccggagaagcuuggugg
aagcccaccuccucaugggacuauaucacaguaaaaagagggaguagucaggacaauagcugu
gagggaaaaugcaacccccugguuuugcaguucacccagaagggaagacaagccucuugggac
ggaccuaagauguggggauugcgacuauaccguacaggauaugacccuaucgcuuuauucacg
gugucccggcagguaucaaccauuacgccgccucaggcaaugggaccaaaccuagucuuaccu
gaucaaaaacccccaucccgacaaucucaaacaggguccaaaguggcgacccagaggccccaa
acgaaugaaagcgccccaaggucuguugcccccaccaccaugggucccaaacggauugggacc
ggagauagguuaauaaauuuaguacaagggacauaccuagccuuaaaugccaccgaccccaac
aaaacuaaagacuguuggcucugccugguuucucgaccacccuauuacgaagggauugcaauc
uuagguaacuacagcaaccaaacaaacccccccccauccugccuaucuacuccgcaacacaaa
cuaacuauaucugaaguaucagggcaaggaaugugcauagggacuguuccuaaaacccaccag
gcuuugugcaauaagacacaacagggacauacaggggcgcacuaucuagccgcccccaacggc
accuauugggccuguaacacuggacucaccccaugcauuuccauggcggugcucaauuggacc
ucugauuuuugugucuuaaucgaauuauggcccagagugacuuaccaucaacccgaauaugug
uacacacauuuugccaaagcugucagguuccgaaga
Feline Calicivirus (VS-FCV) capsid
(SEQ ID NO: 5)
atggctgacgacggatctgtgaccaccccagaacaaggaacaatggtcggaggagtgatt
gccgaacccagcgctcagatgtcaactgcggcggacatggcctccggaaagtcggtggac
tccgagtgggaagccttcttctcgttccacacgtccgtgaactggagcacctccgaaacc
caaggaaagatcctcttcaagcagtccctgggtcccctgctgaacccgtacctggagcac
atcagcaagctgtacgtcgcttggagcgggtcgatcgaagtgcgattttccatctcggga
agcggcgtgttcggtggtaaactggccgccatcgtcgtgccgcctggtgtcgaccctgtc
cagtcaacctccatgctgcagtacccgcacgtcctgttcgacgcaagacaagtggagcca
gtgatcttctccatcccggacctccgcaacagcctgtatcacttgatgtccgataccgat
accacttccctcgtgatcatggtgtacaacgatctgatcaacccgtacgccaatgactcc
aacagctcgggttgcatcgtgaccgtcgaaacgaagcctggcatcgatttcaagtttcat
ctgctgaaaccgcccggatccatgcttactcacgggtccatcccttccgatctgatcccc
aagagctcctccctgtggattgggaaccgccactggaccgatattaccgatttcgtgatt
cggcctttcgtgttccaagccaaccggcacttcgacttcaaccaggagactgccggctgg
tcaactccacggttccgcccattggccgtgactgtgtcgcagtcaaagggagccaagctc
gggaacggcatcgccaccgactacattgtgcctggaatccccgacggatggcctgatact
accatccccaccaagctgacccctaccggagattacgccatcacctcctccgacggcaat
gatattgaaaccaagctggaatacgagaacgcggacgtgattaagaacaacaccaacttc
cgctccatgtatatctgcggaagcctccagagggcttggggcgacaagaagatcagcaac
accgggttcatcactaccggagtgatttctgacaactccatcagcccttcgaacacaatt
gaccagtccaagatcgtggtgtaccaggacaaccatgtcaattcggaggtccagactagc
gacatcactcttgccatcctgggctacaccggaattggagaagaggccataggcgccaac
cgggactccgtcgtgagaatttccgtgcttccggaaactggagcaaggggcggaaatcac
cccatcttctacaaaaattccatgaagctgggctacgtgatctcctccattgacgtgttc
aactcccaaatcctccacacctcgcgccagctgtcactgaacaactacttgttgccccct
gactccttcgcggtgtaccggattattgacagcaacggatcatggttcgacattgggatt
gacagcgatgggttttcattcgtgggcgtgtcgtcatttccaaagctggagtttccgctg
tccgcctcatacatgggcatccagctcgcaaagatccggctggcgtccaacatccggtca
tccatgactaagctgtga
Feline Calicivirus (VS-FCV) capsid
(SEQ ID NO: 6)
MADDGSVTTPEQGTMVGGVIAEPSAQMSTAADMASGKSVDSEWEAFFSFHTSVNWSTSET
QGKILFKQSLGPLLNPYLEHISKLYVAWSGSIEVRFSISGSGVFGGKLAAIVVPPGVDPV
QSTSMLQYPHVLFDARQVEPVIFSIPDLRNSLYHLMSDTDTTSLVIMVYNDLINPYANDS
NSSGCIVTVETKPGIDFKFHLLKPPGSMLTHGSIPSDLIPKSSSLWIGNRHWTDITDFVI
RPFVFQANRHFDFNQETAGWSTPRFRPLAVTVSQSKGAKLGNGIATDYIVPGIPDGWPDT
TIPTKLTPTGDYAITSSDGNDIETKLEYENADVIKNNTNFRSMYICGSLQRAWGDKKISN
TGFITTGVISDNSISPSNTIDQSKIVVYQDNHVNSEVQTSDITLAILGYTGIGEEAIGAN
RDSVVRISVLPETGARGGNHPIFYKNSMKLGYVISSIDVFNSQILHTSRQLSLNNYLLPP
DSFAVYRIIDSNGSWFDIGIDSDGFSFVGVSSFPKLEFPLSASYMGIQLAKIRLASNIRS
SMTKL
Feline Calicivirus (VS-FCV) capsid
(SEQ ID NO: 12)
auggcugacgacggaucugugaccaccccagaacaaggaacaauggucggaggagugauu
gccgaacccagcgcucagaugucaacugcggcggacauggccuccggaaagucgguggac
uccgagugggaagccuucuucucguuccacacguccgugaacuggagcaccuccgaaacc
caaggaaagauccucuucaagcagucccuggguccccugcugaacccguaccuggagcac
aucagcaagcuguacgucgcuuggagcgggucgaucgaagugcgauuuuccaucucggga
agcggcguguucggugguaaacuggccgccaucgucgugccgccuggugucgacccuguc
cagucaaccuccaugcugcaguacccgcacguccuguucgacgcaagacaaguggagcca
gugaucuucuccaucccggaccuccgcaacagccuguaucacuugauguccgauaccgau
accacuucccucgugaucaugguguacaacgaucugaucaacccguacgccaaugacucc
aacagcucggguugcaucgugaccgucgaaacgaagccuggcaucgauuucaaguuucau
cugcugaaaccgcccggauccaugcuuacucacggguccaucccuuccgaucugaucccc
aagagcuccucccuguggauugggaaccgccacuggaccgauauuaccgauuucgugauu
cggccuuucguguuccaagccaaccggcacuucgacuucaaccaggagacugccggcugg
ucaacuccacgguuccgcccauuggccgugacugugucgcagucaaagggagccaagcuc
gggaacggcaucgccaccgacuacauugugccuggaauccccgacggauggccugauacu
accauccccaccaagcugaccccuaccggagauuacgccaucaccuccuccgacggcaau
gauauugaaaccaagcuggaauacgagaacgcggacgugauuaagaacaacaccaacuuc
cgcuccauguauaucugcggaagccuccagagggcuuggggcgacaagaagaucagcaac
accggguucaucacuaccggagugauuucugacaacuccaucagcccuucgaacacaauu
gaccaguccaagaucgugguguaccaggacaaccaugucaauucggagguccagacuagc
gacaucacucuugccauccugggcuacaccggaauuggagaagaggccauaggcgccaac
cgggacuccgucgugagaauuuccgugcuuccggaaacuggagcaaggggcggaaaucac
cccaucuucuacaaaaauuccaugaagcugggcuacgugaucuccuccauugacguguuc
aacucccaaauccuccacaccucgcgccagcugucacugaacaacuacuuguugcccccu
gacuccuucgcgguguaccggauuauugacagcaacggaucaugguucgacauugggauu
gacagcgauggguuuucauucgugggcgugucgucauuuccaaagcuggaguuuccgcug
uccgccucauacaugggcauccagcucgcaaagauccggcuggcguccaacauccgguca
uccaugacuaagcuguga
Feline Calicivirus (F9-like) capsid
(SEQ ID NO: 7)
atgactgccccggaacaaggaacgatggtcggaggagtgattgcagaaccgtcagcacag
atgtccaccgctgccgacatggccactggaaagagcgtggactccgaatgggaagccttc
ttctccttccacacttcggtcaactggtcgactagcgaaacccaggggaagattttgttc
aagcaatccctcggccctctgctgaacccctacctggagcatctggccaagctgtacgtg
gcatggtcgggcagcatcgaagtgcgctttagcatttccggctccggagtgttcggggga
aagcttgctgccattgtcgtgccgccaggagtggacccggtgcagtccacttctatgctc
caatacccgcatgtcctgttcgacgccagacaggtggagcctgtgatcttttgcctgccg
gatctcaggtccaccctgtatcacctcatgtccgacaccgacaccacctcgctcgtgatc
atggtgtacaacgacctgatcaacccctacgctaacgacgccaacagctcaggttgcatt
gtgactgtcgaaaccaagccaggccctgacttcaagtttcatttgctgaagccgcccggt
tccatgctgacccacggctcgatcccatccgacctgatccccaagacgagctccctgtgg
atcggaaaccgctactggtccgatattaccgacttcgtgatcagaccattcgtgttccaa
gccaaccgccatttcgacttcaaccaggaaaccgcaggatggtcgacccctcgattccgc
ccgatttcagtgaccatcaccgaacagaacggcgcgaagctgggaattggcgtggcgacc
gactacatcgtgccgggaatcccggatggatggcctgatacgaccattcccggggagctg
atccctgccggggactacgccatcaccaacggtactggaaacgacatcaccactgccacc
ggttacgacaccgccgacatcataaagaacaacaccaacttcagaggaatgtacatttgc
ggctccctgcaacgcgcttggggtgacaaaaagatctcgaacactgccttcatcacaaca
gcgactctggacggcgataacaacaacaagatcaatccttgtaataccatcgaccagtcc
aaaatcgtggtgttccaggataaccacgtgggaaagaaggcgcagacctccgacgacact
ctggcgctgcttggctacaccgggatcggcgagcaggccattggaagcgatcgggatcgg
gtcgtgcggatctccaccctccccgagactggagcaaggggaggcaaccaccccatcttt
tacaaaaacagcattaagctcggatacgtcatccgctccatcgatgtgttcaactctcaa
atcctgcacacttcgcggcagctgtccctgaaccactacctcttgccgcccgactccttc
gccgtctaccggatcattgattcgaacgggagctggttcgacatcggcattgatagcgat
ggcttctcgtttgtgggcgtgtcgggcttcgggaagctggagttcccactgagcgcctca
tacatgggtatccagctggccaagatcaggctggcctccaacatccgctcacctatgact
aagctgtga
Feline Calicivirus (F9-like) capsid
(SEQ ID NO: 8)
MTAPEQGTMVGGVIAEPSAQMSTAADMATGKSVDSEWEAFFSFHTSVNWSTSETQGKILF
KQSLGPLLNPYLEHLAKLYVAWSGSIEVRFSISGSGVFGGKLAAIVVPPGVDPVQSTSML
QYPHVLFDARQVEPVIFCLPDLRSTLYHLMSDTDTTSLVIMVYNDLINPYANDANSSGCI
VTVETKPGPDFKFHLLKPPGSMLTHGSIPSDLIPKTSSLWIGNRYWSDITDFVIRPFVFQ
ANRHFDFNQETAGWSTPRFRPISVTITEQNGAKLGIGVATDYIVPGIPDGWPDTTIPGEL
IPAGDYAITNGTGNDITTATGYDTADIIKNNTNFRGMYICGSLQRAWGDKKISNTAFITT
ATLDGDNNNKINPCNTIDQSKIVVFQDNHVGKKAQTSDDTLALLGYTGIGEQAIGSDRDR
VVRISTLPETGARGGNHPIFYKNSIKLGYVIRSIDVFNSQILHTSRQLSLNHYLLPPDSF
AVYRIIDSNGSWFDIGIDSDGFSFVGVSGFGKLEFPLSASYMGIQLAKIRLASNIRSPMT
KL
Feline Calicivirus (F9-like) capsid
(SEQ ID NO: 13)
augacugccccggaacaaggaacgauggucggaggagugauugcagaaccgucagcacag
auguccaccgcugccgacauggccacuggaaagagcguggacuccgaaugggaagccuuc
uucuccuuccacacuucggucaacuggucgacuagcgaaacccaggggaagauuuuguuc
aagcaaucccucggcccucugcugaaccccuaccuggagcaucuggccaagcuguacgug
gcauggucgggcagcaucgaagugcgcuuuagcauuuccggcuccggaguguucggggga
aagcuugcugccauugucgugccgccaggaguggacccggugcaguccacuucuaugcuc
caauacccgcauguccuguucgacgccagacagguggagccugugaucuuuugccugccg
gaucucagguccacccuguaucaccucauguccgacaccgacaccaccucgcucgugauc
augguguacaacgaccugaucaaccccuacgcuaacgacgccaacagcucagguugcauu
gugacugucgaaaccaagccaggcccugacuucaaguuucauuugcugaagccgcccggu
uccaugcugacccacggcucgaucccauccgaccugauccccaagacgagcucccugugg
aucggaaaccgcuacugguccgauauuaccgacuucgugaucagaccauucguguuccaa
gccaaccgccauuucgacuucaaccaggaaaccgcaggauggucgaccccucgauuccgc
ccgauuucagugaccaucaccgaacagaacggcgcgaagcugggaauuggcguggcgacc
gacuacaucgugccgggaaucccggauggauggccugauacgaccauucccggggagcug
aucccugccggggacuacgccaucaccaacgguacuggaaacgacaucaccacugccacc
gguuacgacaccgccgacaucauaaagaacaacaccaacuucagaggaauguacauuugc
ggcucccugcaacgcgcuuggggugacaaaaagaucucgaacacugccuucaucacaaca
gcgacucuggacggcgauaacaacaacaagaucaauccuuguaauaccaucgaccagucc
aaaaucgugguguuccaggauaaccacgugggaaagaaggcgcagaccuccgacgacacu
cuggcgcugcuuggcuacaccgggaucggcgagcaggccauuggaagcgaucgggaucgg
gucgugcggaucuccacccuccccgagacuggagcaaggggaggcaaccaccccaucuuu
uacaaaaacagcauuaagcucggauacgucauccgcuccaucgauguguucaacucucaa
auccugcacacuucgcggcagcugucccugaaccacuaccucuugccgcccgacuccuuc
gccgucuaccggaucauugauucgaacgggagcugguucgacaucggcauugauagcgau
ggcuucucguuugugggcgugucgggcuucgggaagcuggaguucccacugagcgccuca
uacauggguauccagcuggccaagaucaggcuggccuccaacauccgcucaccuaugacu
aagcuguga

The following examples serve to provide further appreciation of the invention but are not meant in any way to restrict the effective scope of the invention.

### EXAMPLES

### Example 1

### Incorporation of the Coding Sequences for FELV GP85 into the Alphavirus RNA Replicon Particles

### Introduction

RNA viruses have been used as vector-vehicles for introducing vaccine antigens, which have been genetically engineered into their genomes. However, their use to date has been limited primarily to incorporating viral antigens into the RNA virus and then introducing the virus into a recipient host. The result is the induction of protective antibodies against the incorporated viral antigens. Alphavirus RNA replicon particles have been used to encode pathogenic antigens. Such alphavirus replicon platforms have been developed from several different alphaviruses, including Venezuelan equine encephalitis virus (VEE) [Pushko et al., Virology 239:389-401 (1997)], Sindbis (SIN) [Bredenbeek et al., Journal of Virology 67:6439-6446 (1993) the contents of which are hereby incorporated herein in their entireties], and Semliki Forest virus (SFV) [Liljestrom and Garoff, Biotechnology (NY) 9:1356-1361 (1991), the contents of which are hereby incorporated herein in their entireties]. Moreover, alphavirus RNA replicon particles are the basis for several USDA-licensed vaccines for swine and poultry. These include: Porcine Epidemic Diarrhea Vaccine, RNA Particle (Product Code 19U5.P1), Swine Influenza Vaccine, RNA (Product Code 19A5.D0), Avian Influenza Vaccine, RNA (Product Code 1905.D0), and Prescription Product, RNA Particle (Product Code 9PP0.00).

### Alpha Virus RNA Replicon Particle Construction

An amino acid sequence for FeLV gp85 were used to generate codon-optimized (feline codon usage) nucleotide sequences in silico. Optimized sequences were prepared as synthetic DNA by a commercial vendor (ATUM, Newark, CA). Accordingly, a synthetic gene was designed based on the amino acid sequence of gp85. The construct (gp85_wt) was wild-type amino acid sequence [SEQ ID NO: 2], codon-optimized for feline, with flanking sequence appropriate for cloning into the alphavirus replicon plasmid.

The VEE replicon vectors designed to express FELV gp85 were constructed as previously described [see, U.S. Pat. No. 9,441,247 B2; the contents of which are hereby incorporated herein by reference in their entireties], with the following modifications. The TC-83-derived replicon vector “pVEK” [disclosed and described in U.S. Pat. No. 9,441,247 B2] was digested with restriction enzymes AscI and PacI. A DNA plasmid containing the codon-optimized open reading frame nucleotide sequence of the FeLV gp85 genes, with 5′ flanking sequence (5′-GGCGCGCCGCACC-3′) [SEQ ID NO: 9] and 3′ flanking sequence (5′-TTAATTAA-3′), was similarly digested with restriction enzymes AscI and PacI. The synthetic gene cassette was then ligated into the digested pVEK vector, and the resulting clone was re-named “pVHV-FeLV gp85”. The “pVHV” vector nomenclature was chosen to refer to pVEK-derived replicon vectors containing transgene cassettes cloned via the AscI and PacI sites in the multiple cloning site of pVEK.

Production of TC-83 RNA replicon particles (RP) was conducted according to methods previously described [U.S. Pat. No. 9,441,247 B2 and U.S. Pat. No. 8,460,913 B2; the contents of which are hereby incorporated herein by reference]. Briefly, pVHV replicon vector DNA and helper DNA plasmids were linearized with NotI restriction enzyme prior to in vitro transcription using MegaScript T7 RNA polymerase and cap analog (Promega, Madison, WI). Importantly, the helper RNAs used in the production lack the VEE subgenomic promoter sequence, as previously described [Kamrud et al., J Gen Virol. 91 (Pt 7): 1723-1727 (2010)]. Purified RNA for the replicon and helper components were combined and mixed with a suspension of Vero cells, electroporated in 4 mm cuvettes, and returned to OptiPro® SFM cell culture media (Thermo Fisher, Waltham MA). Following overnight incubation, alphavirus RNA replicon particles were purified from the cells and media by passing the suspension through a ZetaPlus BioCap depth filter (3M, Maplewood, MN), washing with phosphate buffered saline containing 5% sucrose (w/v), and finally eluting the retained RP with 400 mM NaCl buffer. Eluted RP were formulated to a final 5% sucrose (w/v), passed through a 0.22 micron membrane filter, and dispensed into aliquots for storage. Titer of functional RP was determined by immunofluorescence assay on infected Vero cell monolayers.

### Example 2

### Comparative Efficacy and Safety of FELV Vaccines in Cats

A vaccine comprising an alphavirus RNA replicon particle (RP) comprising the capsid protein and glycoproteins of the avirulent TC-83 strain of Venezuelan Equine Encephalitis Virus (VEE) and encoding the FeLV viral glycoprotein (gp85), was formulated in 5% sucrose. The liquid vaccine was frozen for storage before use. This vaccine was compared with a commercially available vaccine comprising a recombinant canary pox encoding FeLV, as shown in Table 1 below. Five groups of eight feline subjects were vaccinated either with a single dose at 8-9 weeks, or in a prime/boost regimen of 8-9 weeks of age and then 21 days later. The doses for each experimental vaccinate group is provided in Table 1 below.

TABLE 1
VACCINATION PROTOCOL

| Vaccinate | No. of | | | Vaccination |
|---|---|---|---|---|
| Group | Animals | Vaccine | RP/dose | Days |
| 1 | 8 | RP-FeLV | 4.35 × 10⁸ | 0, 21 |
| 2 | 8 | RP-FeLV | 3.55 × 10⁷ | 0, 21 |
| 3 | 8 | RP-FeLV | 1.5 × 10⁸ | 21 |
| | | | | (one shot) |
| 4 | 8 | PureVax ® ^{#} | Does not apply | 0, 21 |
| 5 | 8 | Placebo | none | 0, 21 |

^{#} A vaccine containing a recombinant canary pox encoding FeLV sold by Merial ®

All cats were subcutaneously vaccinated with 1.0 mL of their respective vaccine regimen. Cats were 8-9 weeks of age at the time of the initial vaccination (including cats in Group 3). The cats of Group 4 were vaccinated at the times provided with the quantity of vaccine as directed on the label of the commercial vaccine. Following the vaccination the cats were observed for adverse reactions to the vaccines by observing the general health daily, as well as palpating the site of injection for the two days following each vaccination and twice per week for two weeks following each vaccination. No adverse reactions were observed for any of the vaccines.

All cats were challenged with a virulent culture of FeLV four weeks after the booster vaccination (four weeks after the one-shot vaccination for the Group 3 cats). The cats were challenged on four separate days over one week (study days 49, 52, 54 and 56) by administering 1.0 mL of challenge virus by the oronasal route (0.3 mL in each nostril and 0.4 mL orally). Three weeks after challenge serum samples were collected each week through ten weeks post-challenge. Serum samples were tested by ELISA for the presence of FeLV p27 antigen. An animal is considered infected with FeLV if it is persistently antigenemic. Antigenemia is defined as a positive p27 ELISA result for three consecutive weeks or on five or more occasions during the eight week testing period. An FeLV vaccine must protect 75% of the cats vaccinated with the test product for USDA licensure. In addition, in order for the challenge to be regarded as valid, 80% of the control cats must be persistently antigenemic [see, Shipley et al., JAVMA, Vol. 199, No. 10, (Nov. 15, 1991)]. The results of the challenge are summarized in the Table 2 below.

TABLE 2
VACCINATION AND CHALLENGE

| Treatment | | | % Cats | % Cats |
|---|---|---|---|---|
| Group | Vaccine | RP dose | Antigenemic | Protected |
| 1 | RP-FeLV | 4.35 × 10⁸ | 0% | 100% |
| 2 | RP-FeLV | 3.55 × 10⁷ | 0% | 100% |
| 3 | RP-FeLV | 1.5 × 10⁸ | 13% | 87% |
| | | (one shot)* | | |
| 4 | PureVax ® ^{#} | Does not apply | 43% | 57% |
| 5 | Placebo | Does not apply | 88% | 12% |

^{#} A vaccine containing a recombinant canary pox encoding FeLV sold by Merial ®
*All other groups received a two-dose regimen, see, Table 1 above.

As Table 2 demonstrates, the RP-FeLV vaccines protected 100% of the cats when administered in a two-dose regimen (i.e., primary and booster vaccination) at both doses tested. Moreover, the RP-FeLV vaccine protected 87% of the cats when administered as a single dose. In direct contrast, the commercially available vaccine only protected 57% of the cats, even with a two-dose regimen. In addition, the challenge is regarded as valid because greater than 80% of the control cats were persistently antigenemic [see, Table 2]. Finally, all of the RP-FeLV vaccine formulations were found safe in cats.

### Example 3

### Determination of the Dose Dependence of an RP-FELV Vaccine by Vaccination and Challenge

The RP-FeLV vaccine of Example 2 was formulated in a vaccine formulation that included enzymatically hydrolyzed casein (NZ-amine®), gelatin, and sucrose. The vaccine was then lyophilized. Four groups of ten cats each were vaccinated as summarized in Table 3 below:

TABLE 3
VACCINATION PROTOCOL

| Treatment | No. of | | | Vaccination |
|---|---|---|---|---|
| Group | Animals | Vaccine | RP/dose | Days |
| 1 | 10 | RP-FeLV | 1.1 × 10⁵ | 0, 21 |
| 2 | 10 | RP-FeLV | 2.1 × 10⁶ | 0, 21 |
| 3 | 10 | RP-FeLV | 6.5 × 10⁷ | 0, 21 |
| 4 | 10 | Non-vaccinated | None | NA |
| | | Controls | | |

All cats were vaccinated with 1.0 mL of respective test product, subcutaneously. The cats were 8-9 weeks of age at the time of initial vaccination. Following the vaccination the cats were observed for adverse reactions to the vaccines by observing their general daily health, as well as palpating the site of injection for the two days following each vaccination and twice per week for the two weeks following each vaccination. No adverse reactions to any of the vaccines were observed.

All of the cats were challenged with a virulent culture of FeLV three weeks after the booster vaccination. Cats were challenged on four separate days over one week (study days 42, 45, 47 and 49) by administering 1.0 mL of challenge virus by the oronasal route (0.3 mL in each nostril and 0.4 mL orally). Three weeks after challenge serum samples were collected each week through twelve weeks post-challenge. Serum samples were tested by ELISA for the presence of FeLV p27 antigen. An animal is considered infected with FeLV if it is found to be persistently antigenemic. Antigenemia is defined as a positive p27 ELISA result for three consecutive weeks, or on five or more occasions during the eight-week testing period. For USDA licensure an FeLV vaccine must protect 75% of the cats vaccinated with the test product. For the challenge to be considered valid, 80% of the control cats must be persistently antigenemic [Shipley et al., JAVMA, Vol. 199, No. 10, Nov. 15, 1991]. The results of the challenge are summarized in the Table 4 below:

TABLE 4
DOSE DEPENDENCE OF RP-FELV

| Treatment | | | % Cats | % Cats |
|---|---|---|---|---|
| Group | Vaccine | RP/dose | Antigenemic | Protected |
| 1 | RP-FeLV | 1.1 × 10⁵ | 10% | 90% |
| 2 | RP-FeLV | 2.1 × 10⁶ | 0% | 100% |
| 3 | RP-FeLV | 6.5 × 10⁷ | 0% | 100% |
| 4 | Non-vaccinated | None | 90% | 10% |
| | Controls | | | |

In this study of short term immunity, the minimum protective dose of the RP-FeLV vaccine for 100% protection of the cats was between about 1.0×10⁵ to about 2.0×10⁶ RPs, when administered in a two dose (primary and booster vaccination) regimen. The challenge was valid because at least 80% of the control cats were persistently antigenemic. All RP-FeLV vaccine formulations tested were safe in cats.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, and all molecular weight or molecular mass values, given for nucleic acids or polypeptides are approximate, and are provided for description.

1.-19. (canceled)

20. A method comprising:
(a) administering a primer vaccine to a feline subject, the primer vaccine comprising a first Venezuelan Equine Encephalitis (VEE) alphavirus RNA replicon particle (RP) that encodes a first feline leukemia virus (FeLV) glycoprotein 85 (gp85) or an antigenic fragment thereof, and a first pharmaceutically acceptable carrier, and
(b) administering a booster vaccine to the feline subject, the booster vaccine comprising a second VEE alphavirus RNA RP that encodes a second FeLV gp85 or an antigenic fragment thereof, and a second pharmaceutically acceptable carrier.

21. The method of claim 20, wherein the booster vaccine is administered at least 21 days after the primer vaccine is administered.

22. The method of claim 21, wherein the booster vaccine is administered between about 21 days and about 1 year after the primer vaccine is administered.

23. The method of claim 20, wherein the booster vaccine and the primer vaccine are administered via the same route of administration.

24. The method of claim 23, wherein the primer vaccine and the booster vaccine are both administered via subcutaneous injection or oral administration.

25. The method of claim 20, wherein the primer vaccine, or the booster vaccine, or both the primer vaccine and the booster vaccine, comprise less than about 2.0×10⁶ alphavirus RNA RPs, or between about 1.0×10⁵ to about 2.0×10⁶ alphavirus RNA RPs.

26. The method of claim 20, wherein the first FeLV gp85 encoded in the first alphavirus RNA RP present in the primer vaccine is from a different strain of FeLV compared to that of the second FLV gp85 encoded in the second alphavirus RNA RP present in the booster vaccine.

27. The method of claim 20, wherein the first VEE alphavirus RNA RP in the primer vaccine, or the second VEE alphavirus RNA RP in the booster vaccine, or both the first VEE alphavirus RNA RP and the second VEE alphavirus RNA RP, further comprise at least one of:
(a) a nucleic acid construct that encodes at least one non-FeLV antigen for eliciting protective immunity to a non-FeLV pathogen, or
(b) a nucleic acid construct that encodes a feline calicivirus (FCV) antigen which originates from a virulent systemic feline calicivirus or an antigenic fragment thereof, or
(c) a nucleic acid construct that encodes a feline calicivirus (FCV) antigen which originates from a classical (F9-like) feline calicivirus or an antigenic fragment thereof.

28. The method of claim 20, wherein the primer vaccine, or the booster vaccine, or both the primer vaccine and the booster vaccine, are non-adjuvanted, or do not comprise an adjuvant.

29. The method of claim 20, wherein the primer vaccine, or the booster vaccine, or both the primer vaccine and the booster vaccine, further comprise at least one non-FeL V antigen for eliciting protective immunity to a non-FeL V feline pathogen.

30. The method of claim 29, wherein the non-FeLV feline pathogen is selected from the group consisting of feline herpesvirus (FHV), feline calicivirus (FCV), feline pneumovirus (FPN), feline parvovirus (FPV), feline infectious peritonitis virus (FIPV), feline immunodeficiency virus, borna disease virus (BDV), feline influenza virus, feline panleukopenia virus (FPLV), feline coronavirus (FCOV), feline rhinotracheitis virus (FVR), Chlamydophila felis, and any combination thereof.

31. The method of claim 30, wherein the non-FeLV antigen is a killed or attenuated non-FeLV antigen selected from the group consisting of a killed or attenuated feline herpesvirus (FHV), a killed or attenuated feline calicivirus (FCV), a killed or attenuated feline pneumovirus (FPN), a killed or attenuated feline parvovirus (FPV), a killed or attenuated feline infectious peritonitis virus (FIPV), a killed or attenuated feline immunodeficiency virus, a killed or attenuated borna disease virus (BDV), a killed or attenuated feline influenza virus, a killed or attenuated feline panleukopenia virus (FPLV), a killed or attenuated feline coronavirus (FCoV), a killed or attenuated feline rhinotracheitis virus (FVR), a killed or attenuated Chlamydophila felis, and any combination thereof.

32. The method of claim 31, wherein the attenuated non-FeLV antigen is a modified live feline pathogen selected from the group consisting of a modified live Chlamydophila felis, a modified live feline rhinotracheitis Virus (FVR), a modified live feline calicivirus (FCV), a modified live feline panleukopenia virus (FPL), a modified live feline herpesvirus (FHV), a modified live feline pneumovirus (FPN), a modified live feline parvovirus (FPV), a modified live feline infectious peritonitis virus (FIPV), a modified live feline immunodeficiency virus, a modified live borna disease virus (BDV), a modified live feline coronavirus (FCOV), a modified live feline influenza virus, and any combination thereof.

33. The method of claim 20, wherein the primer vaccine, or the booster vaccine, or both the primer vaccine and the booster vaccine, further comprise an alphavirus RNA RP comprising a nucleotide sequence encoding at least one protein antigen or an antigenic fragment thereof that originates from a non-FeL V antigen.

34. The method of claim 33, wherein the protein antigen or an antigenic fragment thereof originates from a non-FeLV feline pathogen selected from the group consisting of feline herpesvirus (FHV), feline calicivirus (FCV), feline pneumovirus (FPN), feline parvovirus (FPV), feline infectious peritonitis virus (FIPV), feline immunodeficiency virus, borna disease virus (BDV), feline influenza virus, feline panleukopenia virus (FPLV), feline coronavirus (FCoV), feline rhinotracheitis virus (FVR), Chlamydophila felis, and any combination thereof.

35. The method of claim 20, wherein the first FeLV gp85, the second FeLV gp85, or both the first FeLV gp85 and the second FeLV gp85, comprise an amino acid sequence having at least 95% identity to the amino acid sequence of SEQ ID NO:2 or SEQ ID NO:4.

36. The method of claim 20, wherein the antigenic fragment of the first FeLV gp85 or the antigenic fragment of the second FeLV gp85 is gp70, or gp45, or an antigenic fragment of gp70 or gp45.

37. The method of claim 20, wherein the primer vaccine is administered to the feline subject at about 8 weeks of age.

38. An alpha RNA replicon particle (RP) comprising a heterologous nucleic acid sequence, the heterologous nucleic acid sequence having at least 95% sequence identity to SEQ ID NO: 1 or SEQ ID NO:10, wherein the RP is a Venezuelan Equine Encephalitis (VEE) RP.

39. The RP of claim 38, wherein the VEE RP is TC-83 VEE RP.

40. The RP of claim 39, wherein the heterologous nucleic acid sequence further comprises a nucleic acid that encodes one or more non-FeLV antigens.
